(19) United States
(12) Patent Application Publication
(10) Pub. No.: US 2026/0308551 A1
(43) Pub. Date: Oct. 8, 2026

(54) MB2CAS12A VARIANTS WITH ENHANCED EFFICIENCY

(71) Applicant: Syngenta Crop Protection AG, Basel (CH)

(72) Inventors: Lizhao Geng, Beijing (CN); Jianping Xu, Beijing (CN); Wan Shi, Beijing (CN)

(73) Assignee: Syngenta Crop Protection AG, Basel (CH)

(21) Appl. No.: 19/149,718

(22) PCT Filed: Jan. 24, 2024
(86) PCT No.: PCT/US2024/012699

§ 371 Date: Jul. 21, 2025

(30) Foreign Application Priority Data
Jan. 27, 2023 (WO) PCT/CN2023/073490

(51) Int. Cl. C12N 9/22; C12N 15/11; C12N 15/82

(52) U.S. Cl. CPC C12N 9/226; C12N 15/11; C12N 15/8213; C12N 2310/20

(57) ABSTRACT

Described herein are Cas 12a mutants from Moraxella bovoculi AAX08 and methods for their use. Mb2Cas12a mutants may comprise single amino acid substitutions, multiple amino acid substitutions, domain swaps, or all the above. These mutants have enhanced DNA cleavage activities in plants compared to the wild-type Moraxella bovoculi AAX08 Cas12a enzyme.

### CLAIM FOR PRIORITY

This application claims priority under 35 U.S.C. § 119 to PCT Application No. PCT/CN2023/073490, filed Jan. 27, 2023, the contents of which are incorporated herein by reference in their entirety.

### FIELD OF THE INVENTION

Described are Cas12a mutants from Moraxella bovoculi AAX08 and methods for use thereof. These mutants have enhanced DNA cleavage activities in plants compared to the wild-type enzyme.

### SEQUENCE LISTING

This application is accompanied by a sequence listing entitled “Mb2Cas12a Variants with Enhanced Efficiency_ST26.xml,” created Nov. 2, 2022, which is approximately 967 kilobytes in size. This sequence listing is incorporated herein by reference in its entirety. This sequence listing is submitted herewith via EFS-Web, and is in compliance with 37 C.F.R. § 1.824 (a) (2)-(6) and (b).

### BACKGROUND

Mb2Cas12a from Moraxella bovoculi AAX08 has demonstrated in planta genome editing capability (Zhang et al., 2021), however the nuclease activity is lower than other Cas12a orthologs widely used in plant or mammalian cell, such as LbCas12a, AsCas12a or FnCas12a (Zetsche et al., 2020; Zhang et al., 2021). With its distinct property of high performance at lower temperature and potential to recognize a shorter PAM, Mb2Cas12a needs to have improved nuclease activity in plants.

### SUMMARY

Therefore, there is a need for a mutant Mb2Cas12a polypeptide variant of the wildtype. This variant may comprise at least one amino acid substitution introduced into a wild-type Mb2Cas12a polypeptide sequence of SEQ ID NO: 1. This substitution can occur at the following positions: D172, F357, F547, A742, E797, Y819, E913, 1914, L917, N918, V921, H939, and/or Y1172. Specific substitutions include: D172R, D172K, F357W, F547Y, A742S, E797A, Y819F, 1914K, L917V, N918A, N918K, V921K, V921Q, H939Q, and Y1172N. Specific sequences of desirable variants of Mb2Cas 12a include SEQ ID NOs: 3, 5, 26, 35, 57, 58, 64, 76, 80, 85, 86, 87, 88, or 127.

An alternative variant can be obtained by swapping a domain from Mb2Cas12a with an orthologous domain from another Cas12a peptide. The domain swap can occur at the following domains: WED-MR1, WED-MR2, BH, Up-seq, and Nuc. The WED-MR1 domain can be replaced with SEQ ID NOs: 24, 29, or 30. The WED-MR2 domain can be replaced with SEQ ID NOs: 25, 32, or 33. The BH domain can be replaced with SEQ ID NOs: 40, 41, or 42. The Up-seq domain can be replaced with SEQ ID NOs: 59, 60, 61, 62, or 63. The Nuc domain can be replaced with SEQ ID NOs: 65, 66, and 67. Domain swap variants of Mb2Cas12a can be include SEQ ID NOs: 37, 59, 60, 61, 62, 63, 74, 75, 77, or 78.

The mutant Mb2Cas12a polypeptide can also comprise an amino acid substitution and a domain swap. Examples of such mutants include SEQ ID NOs: 36, 89, 90, 91, 92, 93, 94, 95, 96, 97, 98, 99, 100, 101, 102, 103, 104, 105, 106, and 107.

These mutants are useful in methods of editing plants, when the mutants described here are used to contact plant genomes. Guide RNA(s), such as those found in SEQ ID NOs: 18-21, may also be used in these methods. Practicing these methods will result in obtaining an edited plant. Constructs and plasmids encoding these mutant Mb2Cas12a polypeptides may be used to express the desired mutants in appropriate organisms and/or tissues. These organisms may be non-human cells, such as plant cells or tissues.

### BRIEF DESCRIPTION OF THE SEQUENCES IN THE SEQUENCE LISTING

SEQ ID NO: 1 is the amino acid sequence of wildtype Mb2Cas12a.

SEQ ID NO: 2 is the nucleotide sequence encoding the amino acid sequence of wildtype Mb2Cas12a.

SEQ ID NO: 3 is the amino acid sequence of Mb2Cas 12a comprising a D172R mutation.

SEQ ID NO: 4 is the nucleotide sequence encoding the amino acid sequence of Mb2Cas12a comprising a D172R mutation.

SEQ ID NO: 5 is the amino acid sequence of Mb2Cas12a comprising a D172K mutation.

SEQ ID NO: 6 is the nucleotide sequence encoding the amino acid sequence of Mb2Cas12a comprising a D172K mutation.

SEQ ID NO: 7 is the nucleotide sequence for a sugarcane ubiquitin promoter (“prSoUbi4-02”).

SEQ ID NO: 8 is the amino acid sequence for an SV40 nuclear localization signal (“xSV40NLS-06”).

SEQ ID NO: 9 is the amino acid sequence for a flexible peptide linker of 30-amino acids having the repeated motif (GGGGS) 6 (“xLinker-06”).

SEQ ID NO: 10 is the amino acid sequence for a short linker of 8-amino acids having the repeated motif (SGGS) 2 (“xSGGSlinker-02”).

SEQ ID NO: 11 is the nucleotide sequence of an Agrobacterium tumefaciens nopaline synthase gene terminator (“tNOS-05-01”).

SEQ ID NO: 12 is the nucleotide sequence of a ribozyme (“rHH-05”).

SEQ ID NO: 13 is the nucleotide sequence of a ribozyme (“rHDV-01”).

SEQ ID NO: 14 is the nucleotide sequence of the crRNA array, inclusive of the ribozymes, targeting four maize genes (“rMb2gRNACas12aZmWxyl-01, rMb2gRNACas12aZmBX9-A, rMb2gRNACas12aZmGL2-01, rMb2gRNACas12aZmBINa”).

SEQ ID NO: 15 is the nucleotide sequence of construct 26411.

SEQ ID NO: 16 is the nucleotide sequence of construct 26363.

SEQ ID NO: 17 is the nucleotide sequence of construct 26410.

SEQ ID NO: 18 is the nucleotide sequence encoding the guide RNA portion which hybridizes to target gene ZmWx1.

SEQ ID NO: 19 is the nucleotide sequence encoding the guide RNA portion which hybridizes to target gene ZmBx9.

SEQ ID NO: 20 is the nucleotide sequence encoding the guide RNA portion which hybridizes to target gene ZmGL2.

SEQ ID NO: 21 is the nucleotide sequence encoding the guide RNA portion which hybridizes to target gene ZmBINa.

SEQ ID NO: 22 is the amino acid sequence of WED-MR1-Mb2 (identical to SEQ ID NO: 31).

SEQ ID NO: 23 is the amino acid sequence of WED-MR2-Mb2 (identical to SEQ ID NO: 32).

SEQ ID NO: 24 is the amino acid sequence of WED-MR1 cognate from LbCas12a.

SEQ ID NO: 25 is the amino acid sequence of WED-MR2 cognate from LbCas12a.

SEQ ID NO: 26 is the amino acid sequence of Mb2Cas 12a comprising the E797A mutation.

SEQ ID NO: 27 is the nucleotide sequence for construct 27731.

SEQ ID NO: 28 is the nucleotide sequence for construct 26840.

SEQ ID NO: 29 is the amino acid sequence of WED-MR1 cognate from AsCas12a.

SEQ ID NO: 30 is the amino acid sequence of WED-MR1 cognate from FnCas12a.

SEQ ID NO: 31 is the amino acid sequence of WED-MR1 cognate from Mb2Cas12a and Mb2Cas12a-22581.

SEQ ID NO: 32 is the amino acid sequence of WED-MR2 cognate from AsCas12a.

SEQ ID NO: 33 is the amino acid sequence of WED-MR2 cognate from FnCas12a.

SEQ ID NO: 34 is the amino acid sequence of WED-MR2 cognate from Mb2Cas12a and Mb2Cas12a-22581.

SEQ ID NO: 35 is the amino acid sequence of Mb2Cas12a comprising the D172R and the E797A mutation.

SEQ ID NO: 36 is the amino acid sequence of Mb2Cas12a comprising the D172R and WED-MR1-Lb.

SEQ ID NO: 37 is the amino acid sequence of Mb2Cas12a comprising WED-MR2-Lb.

SEQ ID NO: 38 is the nucleotide sequence for construct 26841.

SEQ ID NO: 39 is the nucleotide sequence for construct 27493.

SEQ ID NO: 40 is the amino acid sequence of the BH domain of LbCas12a.

SEQ ID NO: 41 is the amino acid sequence of the BH domain of AsCas12a.

SEQ ID NO: 42 is the amino acid sequence of the BH domain of FnCas12a.

SEQ ID NO: 43 is the amino acid sequence of the BH domain of Mb2Cas12a and Mb2Cas12a-22581.

SEQ ID NO: 44 is the amino acid sequence of the Up-seq region of LbCas12a.

SEQ ID NO: 45 is the amino acid sequence of the Up-seq region of AsCas12a.

SEQ ID NO: 46 is the amino acid sequence of the Up-seq region of FnCas12a.

SEQ ID NO: 47 is the amino acid sequence of the Up-seq region of Mb2Cas12a-22581.

SEQ ID NO: 48 is the amino acid sequence of the Up-seq region of Mb2Cas12a.

SEQ ID NO: 49 is the nucleotide sequence for construct 26442.

SEQ ID NO: 50 is the nucleotide sequence for construct 26443.

SEQ ID NO: 51 is the nucleotide sequence for construct 26623.

SEQ ID NO: 52 is the nucleotide sequence for construct 26444.

SEQ ID NO: 53 is the nucleotide sequence for construct 27031.

SEQ ID NO: 54 is the nucleotide sequence for construct 26445.

SEQ ID NO: 55 is the nucleotide sequence for construct 27030.

SEQ ID NO: 56 is the nucleotide sequence for construct 27927.

SEQ ID NO: 57 is the amino acid sequence for Mb2Cas12a V921Q.

SEQ ID NO: 58 is the amino acid sequence for Mb2Cas12a V921K.

SEQ ID NO: 59 is the amino acid sequence for Mb2Cas12a BH1-Lb.

SEQ ID NO: 60 is the amino acid sequence for Mb2Cas12a BH2-Lb.

SEQ ID NO: 61 is the amino acid sequence for Mb2Cas12a BH1-As.

SEQ ID NO: 62 is the amino acid sequence for Mb2Cas12a BH2-As.

SEQ ID NO: 63 is the amino acid sequence for Mb2Cas12a BH3-As.

SEQ ID NO: 64 is the amino acid sequence for Mb2Cas12a L917V+N918K.

SEQ ID NO: 65 is the amino acid sequence of the micro-region in Nuc domain of LbCas12a.

SEQ ID NO: 66 is the amino acid sequence of the micro-region in Nuc domain of AsCas12a.

SEQ ID NO: 67 is the amino acid sequence of the micro-region in Nuc domain of FnCas12a.

SEQ ID NO: 68 is the amino acid sequence of the micro-region in Nuc domain of Mb2Cas12a and Mb2Cas12a-22581.

SEQ ID NO: 69 is the nucleotide sequence for construct 26440.

SEQ ID NO: 70 is the nucleotide sequence for construct 26441.

SEQ ID NO: 71 is the nucleotide sequence for construct 26438.

SEQ ID NO: 72 is the nucleotide sequence for construct 26553.

SEQ ID NO: 73 is the nucleotide sequence for construct 27215.

SEQ ID NO: 74 is the amino acid sequence for Mb2Cas12a Nuc1-Lb.

SEQ ID NO: 75 is the amino acid sequence for Mb2Cas12a Nuc1-As.

SEQ ID NO: 76 is the amino acid sequence for Mb2Cas12a Y1172N.

SEQ ID NO: 77 is the amino acid sequence for Mb2Cas12a Nuc2-Lb.

SEQ ID NO: 78 is the amino acid sequence for Mb2Cas 12a Nuc2-As.

SEQ ID NO: 79 is the nucleotide sequence for construct 26446.

SEQ ID NO: 80 is the amino acid sequence for Mb2Cas12a F357W.

SEQ ID NO: 81 is the nucleotide sequence for construct 27495.

SEQ ID NO: 82 is the nucleotide sequence for construct 27745.

SEQ ID NO: 83 is the nucleotide sequence for construct 27747.

SEQ ID NO: 84 is the nucleotide sequence for construct 27501.

SEQ ID NO: 85 is the amino acid sequence for Mb2Cas12a F547Y.

SEQ ID NO: 86 is the amino acid sequence for Mb2Cas12a A742S.

SEQ ID NO: 87 is the amino acid sequence for Mb2Cas12a Y819F.

SEQ ID NO: 88 is the amino acid sequence for Mb2Cas12a H939Q.

SEQ ID NO: 89 is the amino acid sequence for Mb2Cas12a BH2-As+D172R.

SEQ ID NO: 90 is the amino acid sequence for Mb2Cas12a BH2-As+Nuc1-As+F357W.

SEQ ID NO: 91 is the amino acid sequence for Mb2Cas12a BH2-As+Nuc1-As+F357W+D172R.

SEQ ID NO: 92 is the amino acid sequence for Mb2Cas12a BH2-As+Nuc1-As+F357W+V921K.

SEQ ID NO: 93 is the amino acid sequence for Mb2Cas12a BH2-As+Nuc1-As+F357W+V921K+D172R.

SEQ ID NO: 94 is the amino acid sequence for Mb2Cas12a BH2-As+Nuc1-As+F357W+V921K+E797A.

SEQ ID NO: 95 is the amino acid sequence for Mb2Cas12a BH2-As+Nuc1-As+F357W+V921K+E797A+D172R.

SEQ ID NO: 96 is the amino acid sequence for Mb2Cas12a BH2-As+Nuc1-As+F357W+V921K+E797A+WED-MR1.

SEQ ID NO: 97 is the amino acid sequence for Mb2Cas12a BH2-As+Nuc1-As+F357W+V921K+E797A+WED-MR1+D172R.

SEQ ID NO: 98 is the amino acid sequence for Mb2Cas12a BH2-As+E797A.

SEQ ID NO: 99 is the amino acid sequence for Mb2Cas12a BH2-As+E797A+D172R.

SEQ ID NO: 100 is the amino acid sequence for Mb2Cas12a BH2-As+E797A+F357W.

SEQ ID NO: 101 is the amino acid sequence for Mb2Cas12a BH2-As+E797A+F357W+D172R.

SEQ ID NO: 102 is the amino acid sequence for Mb2Cas12a V921K+E797A.

SEQ ID NO: 103 is the amino acid sequence for Mb2Cas12a V921K+E797A+D172R.

SEQ ID NO: 104 is the amino acid sequence for Mb2Cas12a V921K+E797A+F357W.

SEQ ID NO: 105 is the amino acid sequence for Mb2Cas12a V921K+E797A+F357W+D172R.

SEQ ID NO: 106 is the amino acid sequence for Mb2Cas12a BH2-As+Nuc1-As+F357W+E797A.

SEQ ID NO: 107 is the amino acid sequence for Mb2Cas12a BH2-As+Nuc1-As+F357W+E797A+D172R.

SEQ ID NO: 108 is the nucleotide sequence for construct 27218.

SEQ ID NO: 109 is the nucleotide sequence for construct 27025.

SEQ ID NO: 110 is the nucleotide sequence for construct 27216.

SEQ ID NO: 111 is the nucleotide sequence for construct 27219.

SEQ ID NO: 112 is the nucleotide sequence for construct 27220.

SEQ ID NO: 113 is the nucleotide sequence for construct 27225.

SEQ ID NO: 114 is the nucleotide sequence for construct 27228.

SEQ ID NO: 115 is the nucleotide sequence for construct 27223.

SEQ ID NO: 116 is the nucleotide sequence for construct 27224.

SEQ ID NO: 117 is the nucleotide sequence for construct 27310.

SEQ ID NO: 118 is the nucleotide sequence for construct 27311.

SEQ ID NO: 119 is the nucleotide sequence for construct 27320.

SEQ ID NO: 120 is the nucleotide sequence for construct 27321.

SEQ ID NO: 121 is the nucleotide sequence for construct 27322.

SEQ ID NO: 122 is the nucleotide sequence for construct 27381.

SEQ ID NO: 123 is the nucleotide sequence for construct 27382.

SEQ ID NO: 124 is the nucleotide sequence for construct 27383.

SEQ ID NO: 125 is the nucleotide sequence for construct 27325.

SEQ ID NO: 126 is the nucleotide sequence for construct 27323.

SEQ ID NO: 127 is the amino acid sequence for Mb2Cas12a 1914K+L917V+N918A.

SEQ ID NO: 128 is the nucleotide sequence for construct 27926.

SEQ ID NO: 129 is the nucleotide sequence for an Arabidopsis thaliana EF-1 alpha A1 gene promoter (“prAtEFlaA1”).

SEQ ID NO: 130 is the nucleotide sequence for a Figwort mosaic virus (FMV) enhancer (“eFMV”).

SEQ ID NO: 131 is the nucleotide sequence for a soybean Ubiquitin 1 promoter (“prGmUbi1”).

SEQ ID NO: 132 is the nucleotide sequence encoding for a crRNA targeting A12-fatty acid desaturase II (GmFAD2).

### BRIEF DESCRIPTION OF THE DRAWINGS

FIG. 1 shows the general configuration of the expression cassettes. This same configuration was used for each variant of Mb2Cas12a tested.

### Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used herein, the singular forms “a”, “an” and “the” include plural referents unless the content clearly dictates otherwise. Thus, for example, reference to “an enzyme” optionally includes a combination of two or more such molecules, and the like.

As used herein, “and/or” refers to and encompasses any and all possible combinations of one or more of the associated listed items.

The term “about” as used herein refers to the usual error range for the respective value readily known to the skilled person in this technical field, for example+20%, +10%, or +5%, are within the intended meaning of the recited value.

As used herein, the term “comprising” or “comprise” is open-ended. When used in connection with a subject nucleic acid (or amino acid sequence), it refers to a nucleic acid sequence (or an amino acid sequence) that includes the subject sequence as a part or as its entire sequence.

As used herein, the transitional phrase “consisting essentially of” means that the scope of a claim is to be interpreted to encompass the specified materials or steps recited in the claim and those that do not materially affect the basic and novel characteristic(s) of the claimed matter. Thus, the term “consisting essentially of′ when used in a claim of this disclosure is not intended to be interpreted to be equivalent to “comprising.”

The term “plurality” refers to more than one entity. Thus, a “plurality of individuals” refers to at least two individuals. In some embodiments, the term plurality refers to more than half of the whole. For example, in some embodiments a “plurality of a population” refers to more than half the members of that population.

The term “plant” as used herein refers to any plant at any stage of development, particularly a seed plant. The term “plant cell” as used herein refers to a structural and physiological unit of a plant, comprising a protoplast and a cell wall. The plant cell may be in form of an isolated single cell or a cultured cell, or as a part of higher organized unit such as, for example, plant tissue, a plant organ, or a whole plant. The plant cell may be derived from or part of an angiosperm or gymnosperm. The plant cell may be a monocotyledonous plant cell (e.g., a maize cell, a rice cell, a sorghum cell, a sugarcane cell, a barley cell, a wheat cell, an oat cell, a turf grass cell, or an ornamental grass cell) or a dicotyledonous plant cell (e.g., a tobacco cell, a pepper cell, an eggplant cell, a sunflower cell, a crucifer cell, a flax cell, a potato cell, a cotton cell, a soybean cell, a sugar beet cell, or an oilseed rape cell. The term “plant cell culture” as used herein refers to cultures of plant units such as, for example, protoplasts, cell culture cells, cells in plant tissues, pollen, pollen tubes, ovules, embryo sacs, zygotes and embryos at various stages of development. The term “plant tissue” as used herein refers to a group of plant cells organized into a structural and functional unit. Any tissue of a plant in planta or in culture is included. This term includes, but is not limited to, whole plants, plant organs, plant seeds, tissue culture and any group of plant cells organized into structural and/or functional units. The use of this term in conjunction with, or in the absence of, any specific type of plant tissue as listed above or otherwise embraced by this definition is not intended to be exclusive of any other type of plant tissue. The term “plant part” as used herein refers to a part of a plant, including single cells and cell tissues such as plant cells that are intact in plants, cell clumps and tissue cultures from which plants can be regenerated. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

The terms “polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The terms “nucleic acid” and “polynucleotide” are used interchangeably and as used herein refer to deoxyribonucleic acids (DNA) or ribonucleic acids (RNA) and polymers thereof in either single- or double-stranded form, as well as to both sense and anti-sense strands of RNA, cDNA, genomic DNA, mitochondrial DNA, and synthetic forms and mixed polymers of the above. In higher plants, DNA is the genetic material while RNA is involved in the transfer of information contained within DNA into proteins. A “genome” is the entire body of genetic material contained in each cell of an organism. It is understood that when an RNA is described, its corresponding cDNA is also described, wherein uridine is represented as thymidine. In particular embodiments, a nucleotide refers to a ribonucleotide, deoxynucleotide or a modified form of either type of nucleotide, and combinations thereof. In addition, a polynucleotide disclosed herein may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Nucleotide sequences are “complementary” when they specifically hybridize in solution (e.g., according to Watson-Crick base pairing rules). The term also includes codon-optimized nucleic acids that encode the same polypeptide sequence. It is also understood that nucleic acids can be unpurified, purified, or attached, for example, to a synthetic material such as a bead or column matrix.

The term “corresponding to” in the context of nucleic acid sequences means that when the nucleic acid sequences of certain sequences are aligned with each other, the nucleic acids that “correspond to” certain enumerated positions in the present invention are those that align with these positions in a reference sequence, but that are not necessarily in these exact numerical positions relative to a particular nucleic acid sequence of the invention. Optimal alignment of sequences for comparison can be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. See Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994).

The terms “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. Genes can include both coding and non-coding regions (e.g., introns, regulatory elements, promoters, enhancers, termination sequences and 5′ and 3′ untranslated regions). A gene typically expresses mRNA, functional RNA, or specific protein, including regulatory sequences. Genes may or may not be capable of being used to produce a functional protein. In some embodiments, a gene refers to only the coding region. The term “native gene” refers to a gene as found in nature. The term “chimeric gene” refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and nucleotide sequences of the invention can be considered to be “isolated” as defined above.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence. An “isolated nucleic acid molecule” or “isolated nucleotide sequence” can also include a nucleotide sequence derived from and inserted into the same natural, original cell type, but which is present in a non-natural state, e.g., present in a different copy number, and/or under the control of different regulatory sequences than that found in the native state of the nucleic acid molecule.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not necessarily mean that the preparation is technically pure (homogeneous), but it is sufficiently pure to provide the polypeptide or nucleic acid in a form in which it can be used for the intended purpose.

“Homology dependent repair” or “homology directed repair” or “HDR” refers to a mechanism for repairing ssDNA and double stranded DNA (dsDNA) damage in cells. This repair mechanism can be used by the cell when there is an HDR template with a sequence with significant homology to the injury site. The term “perfect HDR” refers to a situation in which genomic-homology junctions in the replaced allele underwent complete HDR and “imperfect HDR” refers to a situation in which genomic-homology junctions in the replaced allele underwent partial or incomplete HDR. a donor DNA molecule with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor molecule, for example a donor DNA molecule. In some cases, a donor DNA molecule is introduced into a cell. In some cases, at least a segment of a donor DNA molecule integrates into the genome of the cell.

“Microhomology-mediated end joining” or “MMEJ” or “alternative nonhomologous end-joining” (Alt-NHEJ) refers to a form of repairing double-stranded breaks in DNA. This repair mechanism utilizes microhomologous sequences to align the broken strands. “Non-homologous end joining” or “NHEJ” refers to a form of repairing double-stranded breaks in DNA. The double-strand breaks are repaired by direct ligation of the break ends to one another. Generally, no new nucleic acid material is inserted into the site, although some nucleic acid material may be lost or added, resulting in a small deletion or a small insertion.

The proteins provided herein comprise a site-directed polypeptide. A site-directed modifying polypeptide modifies target DNA (e.g., via cleavage or methylation of target DNA) and/or a polypeptide associated with target DNA (e.g., methylation or acetylation of a histone tail). In some embodiments, a site-directed modifying polypeptide interacts with a guide RNA, which is either a single RNA molecule or a RNA duplex of at least two RNA molecules, and is guided to a DNA sequence (e.g. a chromosomal sequence or an extrachromosomal sequence, e.g. an episomal sequence, a minicircle sequence, a mitochondrial sequence, a chloroplast sequence, etc.) by virtue of its association with the guide RNA. In some embodiments, the site-directed polypeptide is a site-directed nuclease, which is able to cleave one or both strands of DNA at a specified target sequence.

The term “cleavage” or “cleaving” refers to breaking of the covalent phosphodiester linkage in the ribosylphosphodiester backbone of a polynucleotide and encompass both single-stranded breaks and double-stranded breaks. Double-stranded cleavage can occur as a result of two distinct single-stranded cleavage events. Cleavage can result in the production of either blunt ends or staggered ends (also known as sticky ends). A “nuclease cleavage site” or “genomic nuclease cleavage site” is a region of nucleotides within which a site-directed nuclease cleaves (e.g., when bound to a proximal binding site). When the polynucleotide is DNA (e.g., genomic DNA), one or both strands can be cleaved at the nuclease cleavage site. Such cleavage by the nuclease enzyme initiates DNA repair mechanisms within the cell, which establishes an environment for homologous recombination to occur.

A site-directed nuclease can be a naturally-occurring site-directed nuclease. Exemplary naturally-occurring site-directed nucleases are known in the art (see for example, Makarova et al., 2017, Cell 168:328-328.el, and Shmakov et al., 2017, Nat Rev Microbiol 15 (3): 169-182, both herein incorporated by reference). In some embodiments, a site-directed nuclease binds a DNA-targeting polynucleotide (e.g., a guide RNA) and is thereby directed to a specific sequence within a target DNA and cleaves the target DNA.

In some embodiments, the site-directed nuclease is modified from its natural sequence (e.g., via mutation or one or more amino acid residues) to change its function. For example, the site-directed nuclease may be modified to be enzymatically inactive. The term “enzymatically inactive” can refer to a site-directed nuclease that can bind to a nucleic acid sequence in a polynucleotide in a sequence-specific manner, but may not cleave a target polynucleotide. An enzymatically inactive site-directed polypeptide can comprise an enzymatically inactive domain (e.g., a nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a wild-type exemplary activity.

In some embodiments, the site-directed nuclease comprises a CRISPR-associated (Cas) protein or a Cas nuclease that functions in a CRISPR (Clustered Regularly Interspaced Short Palindromic Repeats)/Cas system. In bacteria, this system can provide adaptive immunity against foreign DNA (Barrangou, R., et al, “CRISPR provides acquired resistance against viruses in prokaryotes, “Science (2007) 315:1709-1712; Makarova, K.S., et al, “Evolution and classification of the CRISPR-Cas systems,” Nat Rev Microbiol (2011) 9:467-477; Garneau, J. E., et al, “The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39:9275-9282). In a wide variety of organisms including diverse mammals, animals, plants, microbes, and yeast, a CRISPR/Cas system (e.g., modified and/or unmodified) can be utilized as a genome engineering tool. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E2 86; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62; Deltcheva, E., et al, “CRISPR RNA maturation by trans-encoded small RNA and host factor RNase III,” Nature (201 1) 471:602-607). DNA cleavage (e.g., double-strand breaks) can result in DNA break repair which allows for the introduction of gene modification(s) (e.g., nucleic acid editing). DNA break repair can occur via non-homologous end joining (NHEJ), microhomology-mediated end joining (MMEJ), or homology-directed repair (HDR). In some embodiments, donor nucleic acids are used to promote HDR, as detailed below in the “Systems” section. CRISPR-Cas systems have been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31:233-239; Sander, J. D. & Joung, J. K, “CRISPR-Cas systems for editing, regulating and targeting genomes,” Nature Biotechnol. (2014) 32:347-355).

In some embodiments, the site-directed nuclease described herein comprises a Cas protein that forms a complex with a guide nucleic acid, such as a guide RNA (described further below in the “Systems” section). In some embodiments, the site-directed nuclease comprises a Cas protein that forms a complex with a single guide nucleic acid, such as a single guide RNA (sgRNA). In some embodiments, the site-directed nuclease comprises a RNA-binding protein (RBP) optionally complexed with a guide nucleic acid, such as a guide RNA (e.g., sgRNA), which is able to form a complex with a Cas protein. In some instances, RNA-guided Cas proteins recognize DNA targets that are complementary to a portion of the gRNA known as a CRISPR RNA (crRNA) sequence. The target sequence is often referred to as a protospacer, and the part of the crRNA sequence that is complementary to the protospacer is often referred to as a spacer. In order to function (e.g., to cleave DNA), many Cas nucleases also require a specific protospacer adjacent motif (PAM), an approximately 2 to 6 base pair DNA sequence immediately following the protospacer sequence.

Cas proteins from various species (for example, those disclosed in Shmakov et al., 2017, or polypeptides derived therefrom) may require different PAM sequences in the target DNA. Thus, for a particular Cas enzyme of choice, the PAM sequence requirement may be different than the 5′-N GG-3′ sequence (where N is either a A, T, C, or G) known to be required for Cas9 activity. Many Cas9 orthologues from a wide variety of species have been identified, and the proteins share only a few identical amino acids. All identified Cas9 orthologs have the same domain architecture with a central HNH endonuclease domain and a split RuvC/RNaseH domain. Cas9 proteins share 4 key motifs with a conserved architecture; Motifs 1, 2, and 4 are RuvC like motifs, while motif 3 is an HNH-motif. In contrast, Cas12a proteins from various species may have differing PAM sequence requirements compared to the LbCas12a canonical PAM of TTTV.

Any suitable CRISPR/Cas system can be used. A CRISPR/Cas system can be referred to using a variety of naming systems. Exemplary naming systems are provided in Makarova, K.S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

A Cas protein can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas nap hthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Non-limiting examples of Cas proteins include c2c1, C2c2, c2c3, Cas1, Cas1B, Cas2, Cas3, Cas4, Cas5, Cas5e (CasD), Cash, Cas6e, Casof, Cas7, Cas8a, Cas8al, Cas8a2, Cas8b, Cas8c, Cas9 (Csn1 or Csx12), Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, and Cul966, and homologs or modified versions thereof. In some embodiments, the site-directed nuclease of the fusion proteins provided herein comprises a CRISPR-associated nuclease, wherein the CRISPR-associated nuclease is Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, or Cas14. In some embodiments, the CRISPR-associated nuclease is a Cas9 enzyme. In some embodiments, the CRISPR-associated nuclease is a Cas12a enzyme. In some embodiments, the CRISPR-associated nuclease is a nickase or deactivated version of a CRISPR-associated nuclease.

Lachnospiraceae bacterium Cpf1 (LbCpf1) is one of many Cpf1 proteins of a large group. The terms “Cpf1” and “Cas12a” are used interchangeably throughout this disclosure. Cpf1 is a Cas protein. In some embodiments, site-directed nuclease is a catalytically inactive Cas12a from Lachnospiraceae bacterium (“dLbCas12a”). In other embodiments, the site directed nuclease is catalytically active Cas12a from Lachnospiraceae bacterium (“LbCas12a”) or Moraxella bovoculi AAX08_00205 (“Mb2Cas12a”). In some embodiments, the site-directed nuclease domain of the fusion protein is a Cas12a protein from any of Lachnospiraceae bacterium, Acidaminococcus sp., Moraxella bovoculi, Thiomicrospira sp., Moraxella lacunata, Methanomethylophilus alvus, Btyrivibrio sp., or Bacteroidetesoral sp.

As used herein, the term “domain” refers to a discrete, independently folded unit of amino acid residues. Domain size varies, depending on its function and source organism, from about 25-30 amino acid residues to about 300 residues—the average across domains is about 100 residues. Large proteins usually comprise more than one domain. A domain may consist of combinations of motifs. In globular proteins, a motif can be understood as a segment of alpha-helix(es) and/or beta-strand(s) connected by loops to form a recurring pattern. See generally PRINCIPLES OF BIOCHEMISTRY (2d ed.) at 92-96.

A Cas protein can comprise one or more domains. Non-limiting examples of domains include guide nucleic acid recognition and/or binding domains, nuclease domains (e.g., DNase or RNase domains, RuvC, HNH), DNA binding domains, RNA binding domains, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

As used herein, “domain swap” refers to the substitution of one domain with another. The domain substituted can be a recognized domain, a putative domain, a micro-region, or a motif. As used herein, “micro-region” refers to a region at least two amino acid residues within a domain. By way of example and not limitation, a domain which may be subject to a domain swap comprises WED-MR1, WED-MR2, BH, Up-seq, or Nuc, or a combination thereof. By further way of example, one skilled in the art can clone an LbCas12a WED-MR1-encoding domain into the nucleotide sequence encoding Mb2Cas12a, thereby replacing the corresponding Mb2Cas12a WED-MR1 domain.

A Cas protein used herein can be an active variant, inactive variant, or fragment of a wild-type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild-type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type or modified Cas protein or a portion thereof. Variants or fragments can be targeted to a nucleic acid locus in complex with a guide nucleic acid while lacking nucleic acid cleavage activity.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. In some embodiments, double strand break targeting specificity is improved by targeting a nickase to opposite strands at two nearby loci. If a nickase cleaves the single strand at both loci, a double strand break is formed and can be repaired via HR as described herein. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA.

Also provided herein are variants of the polypeptides of this disclosure. Polypeptide variants retain their respective biological activity, unless explicitly noted otherwise. For example, variants of a site-directed nuclease polypeptide retain the biological function of the full length, native sequence site directed nuclease. In another example, variants of the nonspecific end-processing enzyme retain the biological function of the full length, native sequence nonspecific end-processing enzyme.

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-587 (2013); Xie et la. “Adding amino acids to the genetic repertoire,” 9 (6): 548-54 (2005)); and all references cited therein. B and y amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

Also provided is a DNA construct comprising a promoter operably linked to a recombinant nucleic acid encoding a fusion protein or domains thereof as described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription.

The term “promoter” as used herein refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (e.g., forward or reverse) and is capable of functioning even when moved either upstream or downstream from the promoter. The meaning of the term “promoter” includes “promoter regulatory sequences.”

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters”, if the promoters direct RNA synthesis preferentially in certain tissues (RNA synthesis may occur in other tissues at reduced levels). Since patterns of expression of a chimeric gene (or genes) introduced into a plant are controlled using promoters, there is an ongoing interest in the isolation of novel promoters that are capable of controlling the expression of a chimeric gene (or genes) at certain levels in specific tissue types or at specific plant developmental stages.

Certain promoters are able to direct RNA synthesis at relatively similar levels across all tissues of a plant. These are called “constitutive promoters” or “tissue-independent” promoters. Constitutive promoters can be divided into strong, moderate, and weak categories according to their effectiveness to directing RNA synthesis. Since it is necessary in many cases to simultaneously express a chimeric gene (or genes) in different tissues of a plant to get the desired functions of the gene (or genes), constitutive promoters are especially useful in this regard. Though many constitutive promoters have been discovered from plants and plant viruses and characterized, there is still an ongoing interest in the isolation of more novel constitutive promoters, synthetic or native, which are capable of controlling the expression of a chimeric gene (or genes) at different levels and the expression of multiple genes in the same transgenic plant for gene stacking.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of a fusion protein. The cassette may additionally contain at least one additional gene or genetic element to be cotransformed into the cell or organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence (i.e., a nucleic acid sequence that is transcribed into RNA such as mRNA, rRNA, tRNA, snRNA, ncRNA, lncRNA, sense RNA, or antisense RNA, regardless of whether the RNA is then translated to produce a protein) in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided is a vector comprising a recombinant nucleic acid or DNA construct set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. See generally, Sambrook et al. Molecular Cloning: A Laboratory Manual, 4^{th} ed., Cold Spring Harbor Laboratory Press, Cold Spring Harbor, 2012. The vector, for example, can be a plasmid.

Transformation of a cell may be stable or transient. Thus, a transgenic cell, plant cell, plant and/or plant part of the invention can be stably transformed or transiently transformed. Transformation can refer to the transfer of a nucleic acid molecule into the genome of a host cell, resulting in genetically stable inheritance. In some embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or any combination thereof.

Procedures for transforming plants are well known and routine in the art and are described throughout the literature. Non-limiting examples of methods for transformation of plants include transformation via bacterial-mediated nucleic acid delivery (e.g. via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation,, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue. As used herein, the phrase “biolistic transformation” refers to a method of introducing RNA or DNA into cells (e.g., plant cells) directly, in which RNA or DNA is mixed with heavy metal particles (e.g., tungsten or gold) and released into the cell (e.g., the plant cell) using high speed pressure to allow the RNA or DNA to penetrate the cell (e.g., to penetrate the plant cell wall).

The CRISPR/Cas system can also be used to edit the genome of a host cell or organism. As detailed above, the “CRISPR/Cas” system refers to a widespread class of bacterial systems for defense against foreign nucleic acid. Any of the CRISPR/Cas system components described herein may be used to introduce fusion proteins, recombinant nucleic acids, or systems into the genome of a host cell or organism. Methods for CRISPR/Cas system mediated genome editing are known in the art. It will be understood that use of a CRISPR/Cas system for introduction of fusion proteins, recombinant nucleic acids, or systems described herein into the genome of a host cell or organism is different from the particular methods and systems provided herein.

In another aspect, provided herein are systems useful for editing one or more nucleic acids. The systems comprise one or more of the fusion proteins (or recombinant nucleic acids, constructs, vectors, or host cells) described above. In some embodiments, the systems further comprise one or more additional elements that are useful for editing one or more nucleic acids. For example, a system provided herein can further comprise a donor polynucleotide. As another example, a system comprising a fusion protein comprising a Cas nuclease may further comprise one or more guide nucleic acids and/or one or more donor polynucleotide sequences.

In some cases, the systems and methods described herein comprise at least one guide nucleic acid polynucleotide. In some cases, the systems and methods described herein comprise a plurality of guide nucleic acids. In some embodiments, the polynucleotide can be deoxyribonucleic acid (DNA). In some cases, the DNA sequence can be single-stranded or doubled-stranded. In some embodiments, the at least one guide nucleic acid polynucleotide can be ribonucleic acid (guide RNA).

In some embodiments, the nuclease can be complexed with the at least one guide RNA polynucleotide. The at least one guide RNA polynucleotide can comprise a nucleic-acid targeting region that comprises a complementary sequence to a nucleic acid sequence on the targeted polynucleotide such as the targeted genomic loci or genes to confer sequence specificity of nuclease targeting. In some embodiments, the guide nucleic acid is a single guide nucleic acid comprising a crRNA. In some embodiments, the guide nucleic acid is a single guide nucleic acid comprising a crRNA but lacking a tracrRNA. A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence of a targeted polynucleotide can be identified by identifying a protospacer-adjacent motif (PAM) within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The percent complementarity between the nucleic acid-targeting sequence (e.g., a spacer sequence of the at least one guide polynucleotide as disclosed herein) and the target nucleic acid (e.g., a protospacer sequence of the one or more target loci as disclosed herein) can be at least 50%, at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

Guide nucleic acids of the systems of the disclosure can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (a 7-methylguanylate cap (m7G)); a 3′ polyadenylated tail (a 3′ poly(A) tail); a riboswitch sequence (e.g., to allow for regulated stability and/or regulated accessibility by proteins and/or protein complexes); a stability control sequence; a sequence that forms a dsRNA duplex (a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof.

A guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base portion of the nucleotide can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds can be suitable. In addition, linear compounds can have internal nucleotide base complementarity and can therefore fold in a manner as to produce a fully or partially double-stranded compound. Further, within guide nucleic acids, the phosphate groups can commonly be referred to as forming the internucleoside backbone of the guide nucleic acid. The linkage or backbone of the guide nucleic acid can be a 3′ to 5′ phosphodiester linkage.

In some embodiments, the at least one guide RNA polynucleotide of a system or method provided herein can bind to at least a portion of a genome (e.g., a plant genome) or a gene (e.g., a plant gene). In some cases, the at least one guide RNA polynucleotide is capable of forming a complex with a site-directed nuclease to direct the site-directed nuclease to target the portion of a target nucleic acid (e.g., a site in a genome or a gene).

In some embodiments, the systems described herein comprise at least two (e.g., at least three, at least four, at least five, or at least six) different guide RNA polynucleotides that are able to form a complex with a site-directed nuclease.

### DETAILED DESCRIPTION

The following description and examples recite various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

One aspect of the invention is a mutant Mb2Cas12a polypeptide comprising at least one amino acid substitution introduced into a wild-type Mb2Cas12a polypeptide sequence of SEQ ID NO: 1. In one embodiment, the mutant Mb2Cas12a polypeptide comprises at least one amino acid substitution occurs at a position selected from the group consisting of D172, F357, F547, A742, E797, Y819, E913, 1914, L917, N918, V921, H939, and Y1172. In another embodiment, the at least one amino acid substitution is selected from the group consisting of D172R, D172K, F357W, F547Y, A742S, E797A, Y819F, 1914K, L917V, N918A, N918K, V921K, V921Q, H939Q, and Y1172N. In a further embodiment, the polypeptide comprises a sequence selected from the group comprising SEQ ID NOs: 3, 5, 26, 35, 57, 58, 64, 76, 80, 85, 86, 87, 88, and 127.

Another aspect of the invention is a mutant Mb2Cas12a polypeptide comprising at least one domain swap. In one embodiment, the mutant Mb2Cas12a polypeptide comprises a domain swap occurs at a domain selected from the group consisting of WED-MR1, WED-MR2, BH, Up-seq, and Nuc. In another embodiment, the WED-MR1 domain is replaced with a sequence selected from the group consisting of SEQ ID NOs: 24, 29, and 30. In another embodiment, the WED-MR2 domain is replaced with a sequence selected from the group consisting of SEQ ID NOs: 25, 32, and 33. In yet another embodiment, the BH domain is replaced with a sequence selected from the group consisting of SEQ ID NOs: 40, 41, and 42. In another embodiment, the Up-seq domain is replaced with a sequence selected from the group consisting of SEQ ID NOs: 59, 60, 61, 62, and 63. In still yet another embodiment, the Nuc domain is replaced with a sequence selected from the group consisting of SEQ ID NOs: 65, 66, and 67. In a further embodiment, wherein the polypeptide comprises a sequence selected from the group comprising SEQ ID NOs: 37, 59, 60, 61, 62, 63, 74, 75, 77, and 78.

Another aspect of the invention is a mutant Mb2Cas12a polypeptide comprising at least one amino acid substitution and at least one domain swap. In one embodiment, the mutant Mb2Cas12a polypeptide comprises a sequence selected from the group consisting of SEQ ID NOs: 36, 89, 90, 91, 92, 93, 94, 95, 96, 97, 98, 99, 100, 101, 102, 103, 104, 105, 106, and 107.

Another aspect of the invention is a method of editing a plant genome, comprising contacting said plant genome with the mutant Mb2Cas 12a polypeptide of the preceding embodiments. In embodiment, the method of editing a plant genome further comprises guide RNA. In another embodiment, the guide RNA is encoded by a sequence comprising SEQ ID NOs: 18-21.

Another aspect of the invention is an edited plant obtained by the methods of the preceding embodiments.

Another aspect of the invention is a construct or plasmid comprising a polynucleotide sequence encoding for the mutant Mb2Cas 12a polypeptide of the preceding embodiments. One aspect is a non-human cell comprising the construct or plasmid encoding for the mutant Mb2Cas12a polypeptide.

### EXAMPLES

We achieved improved Mb2Cas 12a enzyme activity through protein engineering. Our approaches are rational design and functional domain swapping based on available protein structure of Cas12a orthologs under different states, including MbCas12a-22581 ortholog from same species of Mb2Cas12a (Table 1). The identity of both orthologs is 94.7% of primary amino acid sequences, thus the MbCas12a-22581 serves as a good reference to study Mb2Cas12a functionality, as the latter one does not have reference structure.

TABLE 1
Available Ca12a orthologs and their structures.

| Cas12a | PDB | |
|---|---|---|
| orthologs | identifier | Description |
| MbCas12a- | 6iv6 | Cryo-EM structure of AcrVA5-acetylated MbCas12a/crRNA |
| 22581 | | complex |
| LbCas12a | 5id6 | LbCas12a/RNA complex |
| | 5xus | LbCas12a/crRNA/target DNA complex (TTTA PAM) |
| AsCas12a | 5b43 | AsCas12a/crRNA/target DNA complex |
| FnCas12a | 5NG6 | FnCas12a/crRNA |
| | 5nfv | Inactive FnCas12/pre-crRNA/full-length DNA bounds to R-loop |
| | 5mga | Structure of FnCas12a R-loop complex after DNA cleavage |
| | 6I1K | Inactive FnCas12a/crRNA/dsDNA target |
| | 6i11 | FnCas12a/crRNA/ssDNA target |

“PDB” refers to Protein Data Bank, a public database for archiving information on protein structure. See www.rcsb.org.

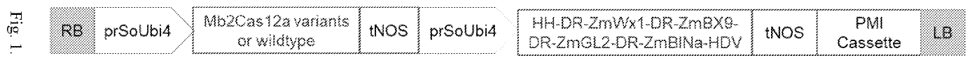

In the examples below, all the constructs comprising the coding sequence of engineered or wildtype Mb2Cas12a for corn transformation have the same configurations of expression cassette and enzyme configuration (FIG. 1). Upstream of the coding sequence is a sugarcane ubiquitin promoter (“prSoUbi4;” SEQ ID NO: 7). The coding sequence of Mb2Cas12a (sequence depends on the variant used) is fused with an SV40 nuclear localization signal (“NLS”) (SEQ ID NO: 8) at the N-terminus via a flexible peptide linker (30-amino acid (GGGGS) 6; SEQ ID NO: 9), and the same peptide linker is fused at the C-terminus with two SV40 NLSs, separated from each other by a short linker (8-amino acid (SGGS) 2; SEQ ID NO: 10). This coding sequence was maize codon optimized and linked to an Agrobacterium tumefaciens nopaline synthase gene terminator (“tNOS;” SEQ ID NO: 11). A crRNA array comprising four crRNAs is flanked by ribozymes HH (SEQ ID NO: 12) 5-prime of the array and HDV (SEQ ID NO: 13) 3-prime of the array, and it was controlled under another set of regulatory components identical to those controlling expression of the selected Mb2Cas12a sequence. This crRNA array expresses four crRNAs targeting four different maize genes: Waxyl (ZmWx1), A UDP-Glucosyltransferase, benzoxazinoid 9 (ZmBX9), Glossy2 (ZmGL2), and BCL2 associated X (ZmBINa). The native Mb2Cas12a mature directed repeat (DR) was adopted as crRNA scaffold for design. The crRNA array is represented by SEQ ID NO: 14, inclusive of the ribozymes.

The constructs were stably transformed to maize immature embryos by a standard transformation protocol (Zhong et al., 2018). Leaf sheath tissue of regenerated plantlets was sampled for DNA extraction, and transgenic plants were identified by TaqMan qPCR assays. Sequencing confirmation of each of the four target sites and analyzing by Taqman qPCR assays were used to determine editing efficiencies of each target site.

### 1. D172 Variants of Mb2Cas12a Improve Editing Efficiency.

To determine whether the Mb2Cas12a D172R or D172K mutation improves enzyme activity in corn, two constructs were built to express each of variant, then transformed into maize for analysis of events.

Table 2 summarizes SDN1 efficiencies at the four target sites. In comparison with the control (wildtype Mb2Cas12 (SEQ ID NO: 1)), the D172R variant (SEQ ID NO: 3) significantly improved the SDN1 efficiency, except for the first crRNA in the array with slight decrease. The D172K variant (SEQ ID NO: 5) significantly improved SDN1 efficiency in all target sites compared to the control, although the range of increase for the last three crRNAs is lower than D172R variant.

TABLE 2
SDN1 editing efficiencies of Mb2Cas12a variants of D172 in maize.

| | Mb2Cas12a | | | | |
|---|---|---|---|---|---|
| Construct | Variant | ZmWx1 | ZmBX9 | ZmGL2 | ZmBINa |
| 26411 | Wildtype | 32.79% | 13.11% | 31.15% | 74.58% |
| | | (20/61) | (8/61) | (19/61) | (44/59) |
| 26363 | D172R | 25.71% | 45.71% | 77.14% | 95.71% |
| | | (18/70) | (32/70) | (54/70) | (67/70) |
| 26410 | D172K | 42.62% | 37.70% | 63.93% | 90.16% |
| | | (26/61) | (23/61) | (39/61) | (55/61) |

Efficiency was measured as a percentage of the number of plants having an indel mutation divided by the total number of transgenic plants.

### 2. Variants of Mutations at WED-III Domain Improve Mb2Cas12a Editing Efficiency.

Cas12a has intrinsic gRNA self-processing capability by processing the 5-prime end of each crRNA from a crRNA array to release individual functional crRNAs. Seeking to improve the gRNA processing capability of Mb2Cas12a, we identified a conserved hydrophilic amino acid residue, glutamic acid, across Cas12a from Moraxella bovoculi species based on the alignment analysis, at position 797 (E797) of Mb2Cas12a. In other Cas12a orthologs, such as Lb, As and Fn, the corresponding position is hydrophobic amino acid, i.e., L807 in AsCas12a, A766 in LbCas12a, and A850 in FnCas12a. We hypothesized the hydrophilic side chain of E797 may affect the local spatial structure due to interaction with surrounding atoms. Therefore, we evaluated E797A mutation with D172R of Mb2Cas12a in stable transformation. Meanwhile, compared to Lb, As, and FnCas12a, one micro-region (MR1) with lower content of acidic/basic amino acid in Moraxella bovoculi species (named WED-MR1; SEQ ID NO: 22) was identified (Table 3). Another micro-region (MR2) with high diversity across Cas12a orthologs (Table 4) was found (named WED-MR2; SEQ ID NO: 23) We made micro-region swapping by replacing WED-MR1 with the cognate LKKEELVV (SEQ ID NO: 24) from LbCas12a into the D172R version of Mb2Cas12a. We further replaced WED-MR2 with the cognate TTTLS (SEQ ID NO: 25) of LbCas12a into the wildtype version of Mb2Cas12a.

TABLE 3
Comparing the amino acids of WED-MR1 across Cas12a
orthologs.

| | Corresponding | |
|---|---|---|
| | Position of | Corresponding |
| | WED-MR1 across | Amino acid sequence |
| | Cas12a | of WED-MR1 across |
| | orthologs | Cas12a orthologs |
| LbCas12a | 751-758 | LKKEELVV (SEQ ID NO: 24) |
| AsCas12a | 794-799 | RMKRMA (SEQ ID NO: 29) |
| FnCas12a | 837-842 | IPKKIT (SEQ ID NO: 30) |
| Mb2Cas12a | 782-789 | LDMNETTI (SEQ ID NO: 31) |
| MbCas12a- | 792-799 | LDMNETTI (SEQ ID NO: 31) |
| 22581 | | |

TABLE 4
Comparing the amino acids of WED-MR2 across Cas12a
orthologs.

| | Corresponding | |
|---|---|---|
| | Position of | Corresponding |
| | WED-MR2 | Amino acid sequence |
| | across Cas12a | of WED-MR2 across |
| | orthologs | Cas12a orthologs |
| LbCas12a | 776-780 | TTTLS (SEQ ID NO: 25) |
| AsCas12a | 817-855 | PIPDTLYQELYDYVNHRLSHDLSDE |
| | | RALLPNVITKEVS |
| | | (SEQ ID NO: 32) |
| FnCas12a | 860-864 | ESVFE (SEQ ID NO: 33) |
| Mb2Cas12a | 807-810 | RQFV (SEQ ID NO: 34) |
| MbCas12a- | 817-820 | RQFV (SEQ ID NO: 34) |
| 22581 | | |

As summarized in Table 5, the SDN1 efficiencies at the four target sites are compared to the in 5 wildtype Mb2Cas12a (SEQ ID NO: 1). The Mb2Cas12a variant with single amino acid mutation E797A (SEQ ID NO: 26) significantly improved the editing efficiency at four target sites. Meanwhile, this mutation also showed the similar contribution under D172R background (i.e., D172R and E797A; SEQ ID NO: 35), which further confirmed the importance of this amino acid for the activity of Mb2Cas12a enzyme. For the construct 26841, coding for Mb2Cas12a variant comprising D172R and WED-MR1 (i.e., SEQ ID NO: 10 36) swapping with the cognate of LbCas12a, the efficiency of the first two crRNAs were enhanced, although slightly decrease was observed for the last crRNA. Comparing to 26411, 27493 harboring WED-MR2-Lb (i.e., SEQ ID NO: 37) swapping with the cognate of LbCas12a improved the efficiency at the first two gRNAs.

TABLE 5
SDN1 editing efficiencies of Mb2Cas12a WED-III
mutation variants in maize

| Construct | Mb2Cas12a Variant | ZmWx1 | ZmBX9 | ZmGL2 | ZmBINa |
|---|---|---|---|---|---|
| 26411 | Wildtype | 32.79% | 13.11% | 31.15% | 74.58% |
| | (SEQ ID NO: 1) | (20/61) | (8/61) | (19/61) | (44/59) |
| 26363 | D172R | 25.71% | 45.71% | 77.14% | 95.71% |
| | (SEQ ID NO: 3) | (18/70) | (32/70) | (54/70) | (67/70) |
| 27731 | E797A | 71.88% | 60.94% | 70.31% | 82.81% |
| | (SEQ ID NO: 26) | (46/64) | (39/64) | (45/64) | (53/64) |
| 26840 | D172R + E797A | 50.00% | 69.74% | 86.84% | 93.42% |
| | (SEQ ID NO: 35) | (38/76) | (53/76) | (66/76) | (71/76) |
| 26841* | D172R + WED- | 41.89% | 66.22% | 78.38% | 86.49% |
| | MR1-Lb | (31/74) | (49/74) | (58/74) | (64/74) |
| | (SEQ ID NO: 36) | | | | |
| 27493 | WED-MR2-Lb | 51.79% | 35.71% | 30.36% | 73.21% |
| | (SEQ ID NO: 37) | (29/56) | (20/56) | (17/56) | (41/56) |

Efficiency was measured as a percentage of the number of plants having an indel mutation divided by the total number of transgenic plants.

### 3. BH Domain Swapping Variants or Lengthening the BH Domain Significantly Improves Mb2Cas12a Activity.

The bridge helix (“BH”) domain is a central helix in the protein that structurally links the REC and the Nuc lobe, which can influence the cleavage accuracy and trimming activity, increase the specificity of Cas12a, and enable the apo enzyme to adopt the closed state, thereby promoting efficient crRNA loading. Disrupting the α-helical nature of the BH in 10 Cas12a alters the trimming activity and cleavage rate (Worle et al., 2021). Alignment of the BH domain across the Cas12a orthologs was conducted (Table. 6). The alignment shows that the length of a-helix in BH domain in MbCas12a-22581 is shorter than that of other orthologs, thus it may influence the enzyme activity.

TABLE 6
Comparing BH domains across Cas12a orthologs.

| | Position of | Amino acid sequence |
|---|---|---|
| | BH domain | of BH domain |
| LbCas12a | 872-886 | YHSLLDKKEKERFEA (SEQ |
| | | ID NO: 40) |
| AsCas12a | 940-957 | YQKKLDNREKERVAARQA |
| | | (SEQ ID NO: 41) |
| FnCas12a | 953-967 | YHDKLAAIEKDRDSA (SEQ |
| | | ID NO: 42) |
| MbCas12a- | 915-926 | YHKILDKREIER (SEQ ID NO: |
| 22581 | | 43) |
| Mb2Cas12a | 905-916 | YHKILDKREIER (SEQ ID NO: |
| | | 43) |

Based on this analysis, we created a series of Mb2Cas12a variants harboring domain swaps of micro region from other orthologs with high enzyme activity as listed in table 8. We also made two variants through rational design with amino acids change to extend the length of alpha helix in BH domain (L917V+N918K and 1914K+L917V+N918A in table 8). Meanwhile, we identified a highly diverse region (“Up-seq”) located in the upstream of BH domain (Table. 7). We also identified a hydrophobic amino acid Val921, which is different in property comparing to the hydrophilic amino acids in AsCas12a (Gln956), LbCas12a (Gln888) or FnCas12a (Lys969) at the same position. The main-chain carbonyl group of Gln956 in the bridge helix can form a hydrogen bond with the side chain of Lys468 in the REC2 domain in AsCas12a (Yamano et al., 2016). Meanwhile, the amide group of side chain forms the interaction with the sugar-phosphate backbone of guide RNA through hydrogen bond. The cognate Val931 in MbCas12a-22581 cannot form such an interaction because the distance between Val931 and sugar-phosphate backbone is beyond 4 Å. Based on the analysis above and the high consistency between Mb2Cas12a and MbCas12a-22581, we created the single amino acid mutation listed in table 8.

TABLE 7
Comparing Up-seq regions across Cas12a orthologs.

| | Position of | Amino acid sequence of |
|---|---|---|
| | Up-seq | Up-seq |
| LbCas12a | 858-871 | EIINNFNGIRIKTD (SEQ ID |
| | | NO: 44) |
| AsCas12a | 934-939 | TIQQFD (SEQ ID NO: 45) |
| FnCas12a | 943-952 | IIGNDRMKTN (SEQ ID NO: |
| | | 46) |
| MbCas12a- | 900-914 | DITTASANGTQMTTP (SEQ |
| 22581 | | ID NO: 47) |
| Mb2Cas12a | 890-904 | DITTASANGTQVTTP (SEQ ID |
| | | NO: 48) |

As summarized in Table 8, the SDN1 efficiencies at the four target sites are compared. In comparison with the control (26411), all those variants significantly improved the SDN1 efficiency at four target sites. The construct 26445 harboring variant BH2-As showed the best performance for editing the 4 targeting sites in corn

TABLE 8
SDN1 editing efficiencies of Mb2Cas12a variants in BH domain in maize.

| | Mb2Cas12a | | | | | |
|---|---|---|---|---|---|---|
| Construct | Variant | Substitution description | ZmWx1 | ZmBX9 | ZmGL2 | ZmBINa |
| 26411 | Wildtype | Control | 32.79% | 13.11% | 31.15% | 74.58% |
| | (SEQ ID | | (20/61) | (8/61) | (19/61) | (44/59) |
| | NO: 1) | | | | | |
| 26442 | V921Q | Single amino acid mutation | 78.26% | 43.48% | 72.46% | 88.41% |
| | (SEQ ID | | (54/69) | (30/69) | (50/69) | (61/69) |
| | NO: 57) | | | | | |
| 26443 | V921K | Single amino acid mutation | 73.44% | 51.56% | 79.69% | 89.06% |
| | (SEQ ID | | (47/64) | (33/64) | (51/64) | (57/64) |
| | NO: 58) | | | | | |
| 26623 | BH1-Lb | Replaced BH with the | 66.22% | 43.24% | 77.03% | 83.78% |
| | (SEQ ID | cognate of LbCas12a | (49/74) | (32/74) | (57/74) | (62/74) |
| | NO: 59) | | | | | |
| 26444 | BH2-Lb | Replaced BH and Up-seq | 77.5% | 47.5% | 87.5% | 87.5% |
| | (SEQ ID | with the cognate of | (31/40) | (19/40) | (35/40) | (35/40) |
| | NO: 60) | LbCas12a | | | | |
| 27031 | BH1-As | Replaced BH with the | 76% | 56% | 80% | 80% |
| | (SEQ ID | cognate of AsCas12a | (19/25) | (14/25) | (20/25) | (20/25) |
| | NO: 61) | | | | | |
| 26445 | BH2-As | Replaced BH and Up-seq | 86.15% | 67.69% | 92.31% | 89.23% |
| | (SEQ ID | with the cognate of | (56/65) | (44/65) | (60/65) | (58/65) |
| | NO: 62) | AsCas12a | | | | |
| 27030 | BH3-As | Replaced Up-seq with the | 67.21% | 47.54% | 78.69% | 77.05% |
| | (SEQ ID | cognate of AsCas12a | (41/61) | (29/61) | (48/61) | (47/61) |
| | NO: 63) | | | | | |
| 27927 | L917V + | amino acid mutation to | 56.45% | 58.06% | 79.03% | 87.10% |
| | N918K | extend the length of alpha | (35/62) | (36/62) | (49/62) | (54/62) |
| | (SEQ ID | helix in BH domain | | | | |
| | NO: 64) | | | | | |
| 27926 | I914K + | Amino acid mutation to | 68.97% | 65.52% | 70.69% | 79.31% |
| | L917V + | extend the length of alpha | (40/58) | (38/58) | (41/58) | (46/58) |
| | N918A | helix in BH domain | | | | |
| | (SEQ ID | | | | | |
| | NO: 127 | | | | | |

### 4. Variants of the Nuc Domain Improve Mb2Cas12a Enzyme Activity.

Endonuclease active site of FnCas12a is located at the interface of the RuvC and Nuc domains and is responsible for the sequential cleavage of target and non-target DNA strands (Swarts et al., 2017). The conserved polar residues Arg1226 and Asp1235 and the partially conserved Ser1228 are clustered in the proximity of the active site of the RuvC domain in AsCas12a, which are the critical residues for the endonuclease activity in the Nuc domain (Yamano et al., 2016). Peptide alignment of Cas12a orthologs shows the surrounding residues at that region are very diverse (Table 9).

TABLE 9
Comparing micro-regions of Nuc domains across
Cas12a orthologs.

| | Position of | |
|---|---|---|
| | micro | Amino acid sequence |
| | region of | of micro region |
| | Nuc domain | of Nuc domain |
| LbCas12a | 1136-1149 | QMRNSITGRTDVDF (SEQ ID NO: |
| | | 65) |
| AsCas12a | 1224-1236 | QMRNSNAATGEDY (SEQ ID NO: |
| | | 66) |
| FnCas12a | 1216-1228 | QMRNSKTGTELDY (SEQ ID NO: |
| | | 67) |
| MbCas12a- | 1179-1191 | ALRYSNASSDEDF (SEQ ID NO: |
| 22581 | | 68) |
| Mb2Cas12a | 1169-1181 | ALRYSNASSDEDF (SEQ ID NO: |
| | | 68) |

Those key residues are in a small region, and we replaced this small region with the cognate sequence of LbCas12a and AsCas12a, respectively (table 10). A point mutation Y1172N in this region was also evaluated (table 10). Meanwhile, considering the entire Nuc domain has little interaction with other domains, according to our structure analysis, we also replaced entire Nuc domain with the cognate sequence of LbCas12a and AsCas12a, respectively (table 10).

TABLE 10
SDN1 editing efficiencies of Mb2Cas12a variants in
Nuc domain in maize.

| Con- | Mb2Cas12a | Substitution | | | | |
|---|---|---|---|---|---|---|
| struct | Variant | description | ZmWx1 | ZmBX9 | ZmGL2 | ZmBINa |
| 26411 | Wildtype | Control | 32.79% | 13.11% | 31.15% | 74.58% |
| | (SEQ ID | | (20/61) | (8/61) | (19/61) | (44/59) |
| | NO: 1) | | | | | |
| 26440 | Nuc1-Lb | Micro region | 15.22% | 4.35% | 15.22% | 34.78% |
| | (SEQ ID | replacement | (7/46) | (2/46) | (7/46) | (16/46) |
| | NO: 74) | | | | | |
| 26441 | Nuc1-As | Micro region | 63.64% | 37.88% | 50% | 78.79% |
| | (SEQ ID | replacement | (42/66) | (25/66) | (33/66) | (52/66) |
| | NO: 75) | | | | | |
| 26438 | Y1172N | Single | 46.88% | 25.37% | 35.82% | 85.07% |
| | (SEQ ID | amino acid | (30/64) | (17/67) | (24/67) | (57/67) |
| | NO: 76) | mutation | | | | |
| 26553 | Nuc2-Lb | Whole Nuc | 44.07% | 42.37% | 22.03% | 76.27% |
| | (SEQ ID | domain | (26/59) | (25/59) | (13/59) | (45/59) |
| | NO: 77) | replacement | | | | |
| 27215 | Nuc2-As | Whole Nuc | 5.26% | 1.75% | 5.26% | 14.04% |
| | (SEQ ID | domain | (3/57) | (1/57) | (3/57) | (8/57) |
| | NO: 78) | replacement | | | | |

As summarized editing efficiency in table 10 shows, three variants, i.e., variant Nuc1-As (in construct 26441) contains the cognate Nuc domain of AsCas12a, variant Y1172N (in construct 26438) a single amino acid mutation, and variant Nuc2-Lb (in construct 26553), a replacement of whole Nuc domain from LbCas12a, significantly improved the editing efficiency.

### 5. The Mb2Cas12a F357W Variant Improves Editing Efficiency.

For Cas12a systems, only twenty base pairs are formed by the interaction between a target DNA sequence and a crRNA, even though our crRNA design uses a spacer length of 23 nucleotides. The partial reason is Trp382 interacts with the twentieth base pair of DNA and crRNA, which lead to the disruption of base pair in position 21 and following. Thus, this amino acid residue is critical for forming the triplex structure of Cas12a-DNA-crRNA.

In AsCas12a, Trp382 forms a stacking interaction with the C20: dG20 pairing in the heteroduplex format (i.e., “dG20” refers to the base at the twentieth position of the DNA target strand; “C20” refers to the base at the twentieth position in the space of the crRNA), thus prevents base pairing between A21 and dT21. In addition, the W382A mutation of AsCas12a decreases enzyme activity (Yamano et al., 2016), which implies the W382 residue contributes to the enzyme performance. A sequence alignment suggests there is a functionally conserved aromatic amino acid at this position across a majority of Cas12a orthologs (Table 11).

TABLE 11
The alignment analysis for Phe357 of Mb2Cas12a.

| | The comparison |
|---|---|
| | of amino acid |
| LbCas12a | W355 |
| AsCas12a | W382 |
| FnCas12a | Y410 |
| MbCas12a-22581 | F370 |
| Mb2Cas12a | F357 |

While performing further analysis on the side chain, the cognate Phe357 of Mb2Cas12a is different from conserved Trp or Tyr residues, which possess hydrogen donor or acceptor atom. Since phenylalanine has no donor or acceptor of hydrogen atom in the side chain, this suggests there is little or no interaction between Phe357 of Mb2Cas 12a and the RNA-DNA heteroduplex, and further implies that Phe357 may reduce enzyme activity. To test this hypothesis, a single residue mutation F357W was constructed for evaluating its functionality in maize.

As summarized in Table 12, the SDN1 efficiencies at the four target sites were compared. In comparison with the control (26411), the F357W variant (26446) improved the SDN1 efficiency at four target sites significantly.

TABLE 12
SDN1 editing efficiencies of F357W variant in REC2 domain in maize.

| Con- | Mb2Cas12a | Substitution | | | | |
|---|---|---|---|---|---|---|
| struct | Variant | description | ZmWx1 | ZmBX9 | ZmGL2 | ZmBINa |
| 26411 | Wildtype | Control | 32.79% | 13.11% | 31.15% | 74.58% |
| | | | (20/61) | (8/61) | (19/61) | (44/59) |
| 26446 | F357W | Single | 56.25% | 31.25% | 64.06% | 81.25% |
| | (SEQ ID | amino acid | (36/64) | (20/64) | (41/64) | (52/64) |
| | NO: 80) | mutation | | | | |

6. Enhancing Interaction of Mb2Cas12a with the 5′ Directed Repeat of Crrna Improves the Sdn1 Efficiency.

In order to cleave a target DNA sequence, a Cas12a protein joins with a crRNA molecule in the form of a ribonucleoprotein (“RNP”) complex. A stronger Cas12a-crRNA interaction, i.e., a more stable Cas12a-crRNA RNP complex, can presumably improve the DNA binding efficiency and the crRNA-dependent DNase activity. Studies of various Cas12a-crRNA complexes suggest Cas12a binds to the pseudoknot structure formed by the 5′ directed repeat (“DR”) in mature crRNAs. We compared the Cas12a-crRNA binary complex structure of LbCas12a with that of MbCas12a-22581. We identified four amino acid residues (see Table 13), each behaving differently between LbCas12a and MbCas12a-22581 in terms of the interaction with DR.

TABLE 13
The amino acid residues interfacing with the pseudoknot.

| LbCas12a | MbCas12a-22581 | Mb2Cas12a |
|---|---|---|
| Y516 | F557 | F547 |
| S713 | A752 | A742 |
| F789 | Y829 | Y819 |
| Q906 | H949 | H939 |

Y516 and S713 of LbCas12a interact with the ribo-phosphate backbone of the DR, while the corresponding residues in MbCas12a-22581 (F557 and A752, respectively) do not interact with DR due to the side chain properties' inability to form hydrogen bonds. Both Q906 of LbCas12a and its counterpart in MbCas12a-22581 (H949) can potentially interact with nucleobases in DR. However, they tend to interact with different nucleobases, and the interaction between H949 of MbCas12a-22581 and U7 of DR (i.e., the uracil at the seventh position of the directed repeat) is more likely interfering with the base pairing in the pseudoknot structure between U7 and A17 (i.e., the adenine at the seventeenth position of the directed repeat). F789 of LbCas12a does not interact with DR, while the counterpart in MbCas12a-22581 (Y829) has a strong potential to form hydrogen bonds with DR due to the additional hydroxyl group in the side chain.

Based on these analyses, we made a series of single-mutation Mb2Cas12a variants by replacing each of the four residues in Mb2Cas12a with the corresponding residue in LbCas12a (Table 14). Similar to the previous examples, nuclear localization signals and polypeptide linkers were added to each variant, and binary vectors were constructed to generate transgenic maize plants and to target four maize genes. Except for the H939Q variant in 27501, the other variants significantly improved the editing efficiency at four target sites.

TABLE 14
SDN1 editing efficiencies comparison in maize.

| Con- | Mb2Cas12a | Substitution | | | | |
|---|---|---|---|---|---|---|
| struct | variant | description | ZmWx1 | ZmBX9 | ZmGL2 | ZmBINa |
| 26411 | Wildtype | Control | 32.79% | 13.11% | 31.15% | 74.58% |
| | | | (20/61) | (8/61) | (19/61) | (44/59) |
| 27495 | F547Y | Single | 76.56% | 50.77% | 58.46% | 90.77% |
| | (SEQ ID | amino acid | (49/64) | (33/65) | (38/65) | (59/65) |
| | NO: 85) | mutation | | | | |
| 27745 | A742S | Single | 75.41% | 67.21% | 72.13% | 85.25% |
| | (SEQ ID | amino acid | (46/61) | (41/61) | (44/61) | (52/61) |
| | NO: 86) | mutation | | | | |
| 27747 | Y819F | Single | 68.97% | 58.33% | 63.33% | 81.67% |
| | (SEQ ID | amino acid | (40/58) | (35/60) | (38/60) | (49/60) |
| | NO: 87) | mutation | | | | |
| 27501 | H939Q | Single | 0.00% | 38.46% | 11.54% | 15.38% |
| | (SEQ ID | amino acid | (0/26) | (10/26) | (3/26) | (4/26) |
| | NO: 88) | mutation | | | | |

### 7. Combinations of Single Region Changes Improves the SDN1 Efficiency at Four Target Sites in Maize.

To further evaluate the synergistic effects of mutations or small peptide replacement validated in the previous experiment for improving enzyme activity, a series constructs of combining various mutations were made as shown in table 15. In comparison to the control (26411), all combinational variants tested improved the efficiency at all target sites, with the sole exception of one variant in 27381 showing a slight decrease at the first target site.

TABLE 15
SDN1 editing efficiencies of combinational variants for comparison with wildtype in maize.

| Construct | Mb2Cas12a Variant | ZmWx1 | ZmBX9 | ZmGL2 | ZmBINa |
|---|---|---|---|---|---|
| 26411 | Wildtype (SEQ ID NO: 1) | 32.79% | 13.11% | 31.15% | 74.58% |
| | | (20/61) | (8/61) | (19/61) | (44/59) |
| 27218 | BH2-As + D172R (SEQ ID NO: 89) | 50.82% | 52.46% | 80.33% | 81.97% |
| | | (31/61) | (32/61) | (49/61) | (50/61) |
| 27025 | BH2-As + Nuc1-As + F357W (SEQ ID NO: 90) | 73.77% | 65.57% | 80.33% | 77.05 |
| | | (45/61) | (40/61) | (49/61) | (47/61) |
| 27216 | BH2-As + Nuc1-As + F357W + D172R (SEQ ID NO: 91) | 47.62% | 71.43% | 85/71% | 85.71% |
| | | (30/63) | (45/63) | (54/63) | (54/63) |
| 27219 | BH2-As + Nuc1-As + F357W + V921K (SEQ ID NO: 92) | 84.75% | 72.88% | 89.83% | 88.14% |
| | | (50/59) | (43/59) | (53/59) | (52/59) |
| 27220 | BH2-As + Nuc1-As + F357W + V921K + D172R | 43.94% | 60.61% | 87.88% | 81.82% |
| | (SEQ ID NO: 93) | (29/66) | (40/66) | (58/66) | (54/66) |
| 27225 | BH2-As + Nuc1-As + F357W + V921K + E797A | 88.00% | 76.00% | 96.00% | 96.00% |
| | (SEQ ID NO: 94) | (22/25) | (19/25) | (24/25) | (24/25) |
| 27228 | BH2-As + Nuc1-As + F357W + V921K + E797A + D172R | 34.78% | 53.62% | 85.51% | 77.94% |
| | (SEQ ID NO: 95) | (24/69) | (37/69) | (59/69) | (53/68) |
| 27223 | BH2-As + Nuc1-As + F357W + V921K + E797A + WED- | 83.02% | 75.47% | 86.79% | 90.57% |
| | MR1 (SEQ ID NO: 96) | (44/53) | (40/53) | (46/53) | (48/53) |
| 27224 | BH2-As + Nuc1-As + F357W + V921K + E797A + WED- | 52.00% | 64.00% | 92.00% | 92.00% |
| | MR1 + D172R (SEQ ID NO: 97) | (13/25) | (16/25) | (23/25) | (23/25) |
| 27310 | BH2-As + E797A (SEQ ID NO: 98) | 82.50% | 72.50% | 90.00% | 92.50% |
| | | (33/40) | (29/40) | (36/40) | (37/40) |
| 27311 | BH2-As + E797A + D172R (SEQ ID NO: 99) | 61.02% | 77.97% | 84.75% | 84.75% |
| | | (36/59) | (46/59) | (50/59) | (50/59) |
| 27320 | BH2-As + E797A + F357W (SEQ ID NO: 100) | 88.89% | 69.44% | 88.89% | 88.89% |
| | | (32/36) | (25/36) | (32/36) | (32/36) |
| 27321 | BH2-As + E797A + F357W + D172R (SEQ ID NO: 101) | 45.83% | 50.00% | 91.67% | 83.33% |
| | | (11/24) | (12/24) | (22/24) | (20/24) |
| 27322 | V921K + E797A (SEQ ID NO: 102) | 79.25% | 58.49% | 88.68% | 90.57% |
| | | (42/53) | (31/53) | (47/53) | (48/53) |
| 27381 | V921K +E797A + D172R (SEQ ID NO: 103) | 28.57% | 46.43% | 82.14% | 89.29% |
| | | (8/28) | (13/28) | (23/28) | (25/28) |
| 27382 | V921K + E797A + F357W (SEQ ID NO: 104) | 76.12% | 56.72% | 76.12% | 79.10% |
| | | (51/67) | (38/67) | (51/67) | (53/67) |
| 27383 | V921K + E797A + F357W + D172R (SEQ ID NO: 105) | 34.38% | 53.13% | 81.25% | 81.25% |
| | | (11/32) | (17/32) | (26/32) | (26/32) |
| 27325 | BH2-As + Nuc1-As + F357W + E797A (SEQ ID NO: 106) | 86.21% | 81.03% | 89.66% | 87.93% |
| | | (50/58) | (47/58) | (52/58) | (51/58) |
| 27323 | BH2-As + Nuc1-As + F357W + E797A + D172R | 54.90% | 74.51% | 86.27% | 84.31% |
| | (SEQ ID NO: 107) | (28/51) | (38/51) | (44/51) | (43/51) |

### 8. Combination of Single Region Changes Improves the SDN1 Efficiency in Soybean.

One of combinational variants was further evaluated in soybean for the effect on the efficiency. Upstream of the Mb2Cas12a coding sequence is an Arabidopsis thaliana EF-1 alpha A1 gene promoter (“prAtEFlaA1;” SEQ ID NO: 129) linked to a Figwort mosaic virus (FMV) enhancer (“eFMV;” SEQ ID NO: 130) to the 5-prime end, And downstream of this coding sequence is an Agrobacterium tumefaciens nopaline synthase gene terminator (“tNOS;” SEQ ID NO: 11). A crRNA (encoded by SEQ ID NO: 132) was controlled by soybean Ubiquitin 1 promoter (“prGmUbi1;” SEQ ID NO: 131) and Agrobacterium tumefaciens nopaline synthase gene terminator (“tNOS;” SEQ ID NO: 11). This crRNA targets A12-fatty acid desaturase II (GmFAD2). The native Mb2Cas12a mature directed repeat (DR) was adopted as crRNA scaffold for design.

The constructs were stably transformed to imbibed mature seeds by a standard transformation protocol (Liang, D. et al. (2023) CRISPR/LbCas12a-Mediated Genome Editing in Soybean. In: Yang, B., Harwood, W., Que, Q. (eds) Plant Genome Engineering. Methods in Molecular Biology, vol 2653. Humana, New York, NY. doi.org/10.1007/978-1-0716-3131-7_3). Leaflet tissue of regenerated plantlets was sampled for DNA extraction, and transgenic plants were identified by TaqMan qPCR assays. Sequencing confirmation of each of the four target sites were used to determine editing efficiencies of each target site.

In comparison with the control (wildtype Mb2Cas12; SEQ ID NO: 1), The combinational variant (SEQ ID NO: 94) significantly improved SDN1 efficiency. This data indicated that the optimizations in maize are applicable for soybean.

TABLE 16
SDN1 editing efficiencies of Mb2Cas12 variant compared to wildtype in
soybean.

| Mb2Cas12a Variant | GmFAD2 |
|---|---|
| Wildtype (SEQ ID NO: 1) | 15.63% |
| | (10/64) |
| BH2-As+Nuc1-As+F357W+V921K+E797A | 51.43% |
| (SEQ ID NO: 94) | (18/35) |

Example 8. Construct annotations.
26411 Annotations:

| Name | Type | Minimum | Maximum | Length | Direction |
|---|---|---|---|---|---|
| bNRB-04 | insertion_seq | 4 | 143 | 140 | reverse |
| bNRB-01-01 | insertion_seq | 101 | 125 | 25 | reverse |
| prSoUbi4-02 | promoter | 217 | 2018 | 1802 | forward |
| u5SoUbi4-02 | 5'UTR | 596 | 660 | 65 | forward |
| iSoUbi4-02 | intron | 661 | 2018 | 1358 | forward |
| Wildtype cMb2Cas12a | CDS | 2037 | 6062 | 4026 | forward |
| xSV40NLS-06 | misc_signal | 2040 | 2060 | 21 | forward |
| xLinker-06 | misc_feature | 2061 | 2150 | 90 | forward |
| xLinker-06 | misc_feature | 5904 | 5993 | 90 | forward |
| xSV40NLS-04 | misc_signal | 5994 | 6014 | 21 | forward |
| xSGGSlinker-02 | misc_feature | 6015 | 6026 | 12 | forward |
| xSGGSlinker-02 | misc_feature | 6027 | 6038 | 12 | forward |
| xSV40NLS-07 | misc_signal | 6039 | 6059 | 21 | forward |
| tNOS-05-01 | terminator | 6070 | 6322 | 253 | forward |
| prSoUbi4-02 | promoter | 6329 | 8130 | 1802 | forward |
| u5SoUbi4-02 | 5'UTR | 6708 | 6772 | 65 | forward |
| iSoUbi4-02 | intron | 6773 | 8130 | 1358 | forward |
| pairing\sequence | misc_feature | 8137 | 8142 | 6 | forward |
| rHH-05 | misc_RNA | 8143 | 8179 | 37 | forward |
| rMb2gRNACas12aZmWxy1-01 | misc_RNA | 8180 | 8222 | 43 | forward |
| rMb2CrRNA-01 | misc_RNA | 8180 | 8199 | 20 | forward |
| ZmWxy1\target | misc_feature | 8200 | 8222 | 23 | forward |
| rMb2gRNACas12aZmBX9-A | misc_RNA | 8223 | 8265 | 43 | forward |
| rMb2CrRNA-01 | misc_RNA | 8223 | 8242 | 20 | forward |
| ZmBX9\target | misc_feature | 8243 | 8265 | 23 | forward |
| rMb2gRNACas12aZmGL2-01 | misc_RNA | 8266 | 8309 | 44 | forward |
| rMb2CrRNA-01 | misc_RNA | 8266 | 8285 | 20 | forward |
| ZmGL2\target | misc_feature | 8286 | 8309 | 24 | forward |
| rMb2gRNACas12aZmBINa | misc_RNA | 8310 | 8352 | 43 | forward |
| rMb2CrRNA-01 | misc_RNA | 8310 | 8329 | 20 | forward |
| ZmBINa\target | misc_feature | 8330 | 8352 | 23 | forward |
| rHDV-01 | misc_RNA | 8353 | 8420 | 68 | forward |
| tNOS-05-01 | terminator | 8421 | 8673 | 253 | forward |
| prUbi1-10 | promoter | 8680 | 10672 | 1993 | forward |
| TSS | misc_feature | 9580 | 9580 | 1 | none |
| iUbi1-02-01 | intron | 9663 | 10672 | 1010 | forward |
| cPMI-01 | CDS | 10685 | 11860 | 1176 | forward |
| tNOS-05-01 | terminator | 11921 | 12173 | 253 | forward |
| bNLB-05 | insertion_seq | 12263 | 12392 | 130 | reverse |
| bNLB-01-01 | insertion_seq | 12298 | 12322 | 25 | reverse |
| cSpec-03 | CDS | 12672 | 13460 | 789 | forward |
| prVirG-01 | promoter | 13555 | 13685 | 131 | forward |
| cVirG-01 | CDS | 13760 | 14485 | 726 | forward |
| cRepA-03 | CDS | 14515 | 15588 | 1074 | forward |
| oVS1-02 | rep_origin | 15631 | 16035 | 405 | forward |
| oCOLE-06 | rep_origin | 16713 | 17519 | 807 | reverse |

26363 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-D172R | CDS | 2037 | 6062 | 4026 |
| xSV40NLS-06 | misc_signal | 2040 | 2060 | 21 |
| xLinker-06 | misc_feature | 2061 | 2150 | 90 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| rHHMb2CrRNA-a | misc_RNA | 8137 | 8179 | 43 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| OVS1-02 | rep_origin | 15631 | 16035 | 405 |
| oCOLE-06 | rep_origin | 16713 | 17519 | 807 |

26410 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-D172K | CDS | 2037 | 6062 | 4026 |
| xSV40NLS-06 | misc_signal | 2040 | 2060 | 21 |
| xLinker-06 | misc_feature | 2061 | 2150 | 90 |
| D172K | misc_RNA | 2664 | 2666 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| rHHMb2CrRNA-a | misc_RNA | 8137 | 8179 | 43 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27731 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-E797A | CDS | 2037 | 6062 | 4026 |
| xSV40NLS-06 | misc_signal | 2040 | 2060 | 21 |
| xLinker-06 | misc_feature | 2061 | 2150 | 90 |
| E797A | mutation | 4539 | 4541 | 3 |
| BH | misc_feature | 4863 | 4898 | 36 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26840 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-D172R+E797A | CDS | 2037 | 6062 | 4026 |
| xSV40NLS-06 | misc_signal | 2040 | 2060 | 21 |
| xLinker-06 | misc_feature | 2061 | 2150 | 90 |
| E797A | misc_feature | 4539 | 4541 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| rHHMb2CrRNA-a | misc_RNA | 8137 | 8179 | 43 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26841 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-D172R+WED-MR1-Lb | CDS | 2037 | 6062 | 4026 |
| XSV0NLS-06 | misc_signal | 2040 | 2060 | 21 |
| xLinker-06 | misc_feature | 2061 | 2150 | 90 |
| clbCas12a-05 | misc_feature | 4497 | 4517 | 21 |
| xLinker-06 | misc_feature | 5904 | 599 | |
| XSV40NLS-04 | misc_signal | 5994 | 6014 | 21 |
| XSGGSlinker-02 | misc_feature | 6015 | 6026 | 12 |
| XSGGSlinker-02 | misc_feature | 6027 | 6038 | 12 |
| XSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| rHHMb2CrRNA-a | misc_RNA | 8137 | 8179 | 43 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27493 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-WED-MR2-Lb | CDS | 2037 | 6065 | 4029 |
| xSV40NLS-06 | misc_signal | 2040 | 2060 | 21 |
| xLinker-06 | misc_feature | 2061 | 2150 | 90 |
| RQFV to TTTLS | misc_feature | 4569 | 4583 | 15 |
| xLinker-06 | misc_feature | 5907 | 5996 | 90 |
| xSV40NLS-04 | misc_signal | 5997 | 6017 | 21 |
| xSGGSlinker-02 | misc_feature | 6018 | 6029 | 12 |
| xSGGSlinker-02 | misc_feature | 6030 | 6041 | 12 |
| xSV40NLS-07 | misc_signal | 6042 | 6062 | 21 |
| tNOS-05-01 | terminator | 6073 | 6325 | 253 |
| prSoUbi4-02 | promoter | 6332 | 8133 | 1802 |
| u5SoUbi4-02 | 5'UTR | 6711 | 6775 | 65 |
| iSoUbi4-02 | intron | 6776 | 8133 | 1358 |
| pairing\sequence | misc_feature | 8140 | 8145 | 6 |
| rHH-05 | misc_RNA | 8146 | 8182 | 37 |
| rMb2gRNACas12aZmWxy1-01 | misc_RNA | 8183 | 8225 | 43 |
| rMb2CrRNA-01 | misc_RNA | 8183 | 8202 | 20 |
| ZmWxy1\target | misc_feature | 8203 | 8225 | 23 |
| rMb2gRNACas12aZmBX9-A | misc_RNA | 8226 | 8268 | 43 |
| rMb2CrRNA-01 | misc_RNA | 8226 | 8245 | 20 |
| ZmBX9\target | misc_feature | 8246 | 8268 | 23 |
| rMb2gRNACas12aZmGL2-01 | misc_RNA | 8269 | 8312 | 44 |
| rMb2CrRNA-01 | misc_RNA | 8269 | 8288 | 20 |
| ZmGL2\target | misc_feature | 8289 | 8312 | 24 |
| rMb2gRNACas12aZmBINa | misc_RNA | 8313 | 8355 | 43 |
| rMb2CrRNA-01 | misc_RNA | 8313 | 8332 | 20 |
| ZmBINa\target | misc_feature | 8333 | 8355 | 23 |
| rHDV-01 | misc_RNA | 8356 | 8423 | 68 |
| tNOS-05-01 | terminator | 8424 | 8676 | 253 |
| prUbi1-10 | promoter | 8683 | 10675 | 1993 |
| TSS | misc_feature | 9583 | 9583 | 1 |
| iUbi1-02-01 | intron | 9666 | 10675 | 1010 |
| cPMI-01 | CDS | 10688 | 11863 | 1176 |
| tNOS-05-01 | terminator | 11924 | 12176 | 253 |
| bNLB-05 | insertion_seq | 12266 | 12395 | 130 |
| bNLB-01-01 | insertion_seq | 12301 | 12325 | 25 |
| cSpec-03 | CDS | 12675 | 13463 | 789 |
| prVirG-01 | promoter | 13558 | 13688 | 131 |
| cVirG-01 | CDS | 13763 | 14488 | 726 |
| cRepA-03 | CDS | 14518 | 15591 | 1074 |
| oVS1-02 | rep_origin | 15634 | 16038 | 405 |
| oCOLE-06 | rep_origin | 16716 | 17522 | 807 |

26442 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-V921Q | CDS | 2037 | 6062 | 4026 |
| xSV40NLS-06 | misc_signal | 2040 | 2060 | 21 |
| xLinker-06 | misc_feature | 2061 | 2150 | 90 |
| V921Q | misc_feature | 4911 | 4913 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26443 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-V921K | CDS | 2037 | 6062 | 4026 |
| xSV40NLS-06 | misc_signal | 2040 | 2060 | 21 |
| xLinker-06 | misc_feature | 2061 | 2150 | 90 |
| V921K | misc_feature | 4911 | 4913 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26623 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH1-Lb | CDS | 2037 | 6062 | 4026 |
| xSV40NLS-06 | misc. | 2040 | 2060 | 21 |
| xLinker-06 | misc fea ure | 2061 | 2150 | 90 |
| BH1-1b | misc. | 4869 | 4904 | 36 |
| xLinker-06 | misc | 5904 | 5993 | 90 |
| xSV40NLS-04 | misc. | 5994 | 6014 | 21 |
| xSGGSlinker-02 | misc. fes ture | 6015 | 6026 | 12 |
| xSGGSlinker-02 | misc. | 6027 | 6038 | 12 |
| xSV40NLS-07 | mist | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 25 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26444 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-Lb | CDS | 2037 | 6059 | 4023 |
| xSV40NLS-06 | misc_signal | 2040 | 2060 | 21 |
| xLinker-06 | misc_feature | 2061 | 2150 | 90 |
| BH2-Lb | misc_feature | 4824 | 4913 | 90 |
| xLinker-06 | misc_feature | 5901 | 5990 | 90 |
| xSV40NLS-04 | misc_signal | 5991 | 6011 | 21 |
| xSGGSlinker-02 | misc_feature | 6012 | 6023 | 12 |
| xSGGSlinker-02 | misc_feature | 6024 | 6035 | 12 |
| xSV40NLS-07 | misc_signal | 6036 | 6056 | 21 |
| tNOS-05-01 | terminator | 6067 | 6319 | 253 |
| prSoUbi4-02 | promoter | 6326 | 8127 | 1802 |
| u5SoUbi4-02 | 5'UTR | 6705 | 6769 | 65 |
| iSoUbi4-02 | intron | 6770 | 8127 | 1358 |
| pairing\sequence | misc_feature | 8134 | 8139 | 6 |
| rHH-05 | misc_RNA | 8140 | 8176 | 37 |
| rMb2gRNACas12aZmWxy1-01 | misc_RNA | 8177 | 8219 | 43 |
| rMb2CrRNA-01 | misc_RNA | 8177 | 8196 | 20 |
| ZmWxy1\target | misc_feature | 8197 | 8219 | 23 |
| rMb2gRNACas12aZmBX9-A | misc_RNA | 8220 | 8262 | 43 |
| rMb2CrRNA-01 | misc_RNA | 8220 | 8239 | 20 |
| ZmBX9\target | misc_feature | 8240 | 8262 | 23 |
| rMb2gRNACas12aZmGL2-01 | misc_RNA | 8263 | 8306 | 44 |
| rMb2CrRNA-01 | misc_RNA | 8263 | 8282 | 20 |
| ZmGL2\target | misc_feature | 8283 | 8306 | 24 |
| rMb2gRNACas12aZmBINa | misc_RNA | 8307 | 8349 | 43 |
| rMb2CrRNA-01 | misc_RNA | 8307 | 8326 | 20 |
| ZmBINa\target | misc_feature | 8327 | 8349 | 23 |
| rHDV-01 | misc_RNA | 8350 | 8417 | 68 |
| tNOS-05-01 | terminator | 8418 | 8670 | 253 |
| prUbi1-10 | promoter | 8677 | 10669 | 1993 |
| TSS | misc_feature | 9577 | 9577 | 1 |
| iUbi1-02-01 | intron | 9660 | 10669 | 1010 |
| CPMI-01 | CDS | 10682 | 11857 | 1176 |
| tNOS-05-01 | terminator | 11918 | 12170 | 253 |
| bNLB-05 | insertion_seq | 12260 | 12389 | 130 |
| bNLB-01-01 | insertion_seq | 12295 | 12319 | 25 |
| cSpec-03 | CDS | 12669 | 13457 | 789 |
| prVirG-01 | promoter | 13552 | 13682 | 131 |
| cVirG-01 | CDS | 13757 | 14482 | 726 |
| cRepA-03 | CDS | 14512 | 15585 | 1074 |
| oVS1-02 | rep_origin | 15628 | 16032 | 405 |
| oCOLE-06 | rep_origin | 16710 | 17516 | 807 |

27031 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH1-As | CDS | 2037 | 6062 | 4026 |
| xSV40NLS-06 | misc_signal | 2040 | 2060 | 21 |
| xLinker-06 | misc_feature | 2061 | 2150 | 90 |
| BH1-As | misc_feature | 4866 | 4916 | 51 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26445 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As | CDS | 2037 | 6035 | 3999 |
| xSV40NLS-06 | misc_signal | 2040 | 2060 | 21 |
| xLinker-06 | misc_feature | 2061 | 2150 | 90 |
| Up-seq-BH2 | misc_feature | 4818 | 4889 | 72 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27030 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH3-As | CDS | 2037 | 6035 | 3999 |
| xSV40NLS-06 | misc_signal | 2040 | 2060 | 21 |
| xLinker-06 | misc_feature | 2061 | 2150 | 90 |
| BH3-As | misc_feature | 4818 | 4835 | 18 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27927 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-L917V+N918K | CDS | 2037 | 6062 | 4026 |
| xSV40NLS-06 | misc_signal | 2040 | 2060 | 21 |
| xLinker-06 | misc_feature | 2061 | 2150 | 90 |
| F547 | mutation | 3789 | 3791 | 3 |
| A742 | mutation | 4374 | 4376 | 3 |
| E797 | mutation | 4539 | 4541 | 3 |
| BH | misc_feature | 4863 | 4898 | 36 |
| L917V | mutation | 4899 | 4901 | 3 |
| N918K | mutation | 4902 | 4904 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26440 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-Nuc1-Lb | CDS | 2037 | 6065 | 4029 |
| xSV40NLS-06 | misc_signal | 2040 | 2060 | 21 |
| xLinker-06 | misc_feature | 2061 | 2150 | 90 |
| alpha helix | misc_feature | 5610 | 5654 | 45 |
| xLinker-06 | misc_feature | 5907 | 5996 | 90 |
| xSV40NLS-04 | misc_signal | 5997 | 6017 | 21 |
| xSGGSlinker-02 | misc_feature | 6018 | 6029 | 12 |
| xSGGSlinker-02 | misc_feature | 6030 | 6041 | 12 |
| xSV40NLS-07 | misc_signal | 6042 | 6062 | 21 |
| tNOS-05-01 | terminator | 6073 | 6325 | 253 |
| prSoUbi4-02 | promoter | 6332 | 8133 | 1802 |
| u5SoUbi4-02 | 5'UTR | 6711 | 6775 | 65 |
| iSoUbi4-02 | intron | 6776 | 8133 | 1358 |
| pairing\sequence | misc_feature | 8140 | 8145 | 6 |
| rHH-05 | misc_RNA | 8146 | 8182 | 37 |
| rMb2gRNACas12aZmWxy1-01 | misc_RNA | 8183 | 8225 | 43 |
| rMb2CrRNA-01 | misc_RNA | 8183 | 8202 | 20 |
| ZmWxy1\target | misc_feature | 8203 | 8225 | 23 |
| rMb2gRNACas12aZmBX9-A | misc_RNA | 8226 | 8268 | 43 |
| rMb2CrRNA-01 | misc_RNA | 8226 | 8245 | 20 |
| ZmBX9\target | misc_feature | 8246 | 8268 | 23 |
| rMb2gRNACas12aZmGL2-01 | misc_RNA | 8269 | 8312 | 44 |
| rMb2CrRNA-01 | misc_RNA | 8269 | 8288 | 20 |
| ZmGL2\target | misc_feature | 8289 | 8312 | 24 |
| rMb2gRNACas12aZmBINa | misc_RNA | 8313 | 8355 | 43 |
| rMb2CrRNA-01 | misc_RNA | 8313 | 8332 | 20 |
| ZmBINa\target | misc_feature | 8333 | 8355 | 23 |
| rHDV-01 | misc_RNA | 8356 | 8423 | 68 |
| tNOS-05-01 | terminator | 8424 | 8676 | 253 |
| prUbi1-10 | promoter | 8683 | 10675 | 1993 |
| TSS | misc_feature | 9583 | 9583 | 1 |
| iUbi1-02-01 | intron | 9666 | 10675 | 1010 |
| cPMI-01 | CDS | 10688 | 11863 | 1176 |
| tNOS-05-01 | terminator | 11924 | 12176 | 253 |
| bNLB-05 | insertion_seq | 12266 | 12395 | 130 |
| bNLB-01-01 | insertion_seq | 12301 | 12325 | 25 |
| cSpec-03 | CDS | 12675 | 13463 | 789 |
| prVirG-01 | promoter | 13558 | 13688 | 131 |
| cVirG-01 | CDS | 13763 | 14488 | 726 |
| cRepA-03 | CDS | 14518 | 15591 | 1074 |
| oVS1-02 | rep_origin | 15634 | 16038 | 405 |
| oCOLE-06 | rep_origin | 16716 | 17522 | 807 |

26441 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-Nuc1-As | CDS | 2037 | 6062 | 4026 |
| xSV40NLS-06 | misc_signal | 2040 | 2060 | 21 |
| xLinker-06 | misc_feature | 2061 | 2150 | 90 |
| alpha helix | misc_feature | 5610 | 5654 | 45 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26438 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-Y1172N | CDS | 2037 | 6062 | 4026 |
| xSV40NLS-06 | misc_signal | 2040 | 2060 | 21 |
| xLinker-06 | misc_feature | 2061 | 2150 | 90 |
| R1171 | misc_feature | 5661 | 5663 | 3 |
| Y1172N | misc_feature | 5664 | 5666 | 3 |
| Junction | misc_feature | 5667 | 5672 | 6 |
| S1173 | misc_feature | 5667 | 5669 | 3 |
| D1180 | misc_feature | 5688 | 5690 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

26553 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-Nuc2-Lb | CDS | 2037 | 6077 | 4041 |
| xSV40NLS-06 | misc_signal | 2040 | 2060 | 21 |
| xLinker-06 | misc_feature | 2061 | 2150 | 90 |
| Nuc2-Lb | misc_feature | 5241 | 5786 | 546 |
| xLinker-06 | misc_feature | 5919 | 6008 | 90 |
| xSV40NLS-04 | misc_signal | 6009 | 6029 | 21 |
| xSGGSlinker-02 | misc_feature | 6030 | 6041 | 12 |
| xSGGSlinker-02 | misc_feature | 6042 | 6053 | 12 |
| xSV40NLS-07 | misc_signal | 6054 | 6074 | 21 |
| tNOS-05-01 | terminator | 6085 | 6337 | 253 |
| prSoUbi4-02 | promoter | 6344 | 8145 | 1802 |
| u5SoUbi4-02 | 5'UTR | 6723 | 6787 | 65 |
| iSoUbi4-02 | intron | 6788 | 8145 | 1358 |
| pairing\sequence | misc_feature | 8152 | 8157 | 6 |
| rHH-05 | misc_RNA | 8158 | 8194 | 37 |
| rMb2gRNACas12aZmWxy1-01 | misc_RNA | 8195 | 8237 | 43 |
| rMb2CrRNA-01 | misc_RNA | 8195 | 8214 | 20 |
| ZmWxy1\target | misc_feature | 8215 | 8237 | 23 |
| rMb2gRNACas12aZmBX9-A | misc_RNA | 8238 | 8280 | 43 |
| rMb2CrRNA-01 | misc_RNA | 8238 | 8257 | 20 |
| ZmBX9\target | misc_feature | 8258 | 8280 | 23 |
| rMb2gRNACas12aZmGL2-01 | misc_RNA | 8281 | 8324 | 44 |
| rMb2CrRNA-01 | misc_RNA | 8281 | 8300 | 20 |
| ZmGL2\target | misc_feature | 8301 | 8324 | 24 |
| rMb2gRNACas12aZmBINa | misc_RNA | 8325 | 8367 | 43 |
| rMb2CrRNA-01 | misc_RNA | 8325 | 8344 | 20 |
| ZmBINa\target | misc_feature | 8345 | 8367 | 23 |
| rHDV-01 | misc_RNA | 8368 | 8435 | 68 |
| tNOS-05-01 | terminator | 8436 | 8688 | 253 |
| prUbi1-10 | promoter | 8695 | 10687 | 1993 |
| TSS | misc_feature | 9595 | 9595 | 1 |
| iUbi1-02-01 | intron | 9678 | 10687 | 1010 |
| cPMI-01 | CDS | 10700 | 11875 | 1176 |
| tNOS-05-01 | terminator | 11936 | 12188 | 253 |
| bNLB-05 | insertion_seq | 12278 | 12407 | 130 |
| bNLB-01-01 | insertion_seq | 12313 | 12337 | 25 |
| cSpec-03 | CDS | 12687 | 13475 | 789 |
| prVirG-01 | promoter | 13570 | 13700 | 131 |
| cVirG-01 | CDS | 13775 | 14500 | 726 |
| cRepA-03 | CDS | 14530 | 15603 | 1074 |
| oVS1-02 | rep_origin | 15646 | 16050 | 405 |
| oCOLE-06 | rep_origin | 16728 | 17534 | 807 |

27215 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-Nuc2-As | CDS | 2037 | 6119 | 4083 |
| xSV40NLS-06 | misc_signal | 2040 | 2060 | 21 |
| xLinker-06 | misc_feature | 2061 | 2150 | 90 |
| Nuc2-As | misc_feature | 5241 | 5828 | 588 |
| xLinker-06 | misc_feature | 5961 | 6050 | 90 |
| xSV40NLS-04 | misc_signal | 6051 | 6071 | 21 |
| xSGGSlinker-02 | misc_feature | 6072 | 6083 | 12 |
| xSGGSlinker-02 | misc_feature | 6084 | 6095 | 12 |
| xSV40NLS-07 | misc_signal | 6096 | 6116 | 21 |
| tNOS-05-01 | terminator | 6127 | 6379 | 253 |
| prSoUbi4-02 | promoter | 6386 | 8187 | 1802 |
| u5SoUbi4-02 | 5'UTR | 6765 | 6829 | 65 |
| iSoUbi4-02 | intron | 6830 | 8187 | 1358 |
| pairing\sequence | misc_feature | 8194 | 8199 | 6 |
| rHH-05 | misc_RNA | 8200 | 8236 | 37 |
| rMb2gRNACas12aZmWxy1-01 | misc_RNA | 8237 | 8279 | 43 |
| rMb2CrRNA-01 | misc_RNA | 8237 | 8256 | 20 |
| ZmWxy1\target | misc_feature | 8257 | 8279 | 23 |
| rMb2gRNACas12aZmBX9-A | misc_RNA | 8280 | 8322 | 43 |
| rMb2CrRNA-01 | misc_RNA | 8280 | 8299 | 20 |
| ZmBX9\target | misc_feature | 8300 | 8322 | 23 |
| rMb2gRNACas12aZmGL2-01 | misc_RNA | 8323 | 8366 | 44 |
| rMb2CrRNA-01 | misc_RNA | 8323 | 8342 | 20 |
| ZmGL2\target | misc_feature | 8343 | 8366 | 24 |
| rMb2gRNACas12aZmBINa | misc_RNA | 8367 | 8409 | 43 |
| rMb2CrRNA-01 | misc_RNA | 8367 | 8386 | 20 |
| ZmBINa\target | misc_feature | 8387 | 8409 | 23 |
| rHDV-01 | misc_RNA | 8410 | 8477 | 68 |
| tNOS-05-01 | terminator | 8478 | 8730 | 253 |
| prUbi1-10 | promoter | 8737 | 10729 | 1993 |
| TSS | misc_feature | 9637 | 9637 | 1 |
| iUbi1-02-01 | intron | 9720 | 10729 | 1010 |
| cPMI-01 | CDS | 10742 | 11917 | 1176 |
| tNOS-05-01 | terminator | 11978 | 12230 | 253 |
| bNLB-05 | insertion_seq | 12320 | 12449 | 130 |
| bNLB-01-01 | insertion_seq | 12355 | 12379 | 25 |
| cSpec-03 | CDS | 12729 | 13517 | 789 |
| prVirG-01 | promoter | 13612 | 13742 | 131 |
| cVirG-01 | CDS | 13817 | 14542 | 726 |
| cRepA-03 | CDS | 14572 | 15645 | 1074 |
| oVS1-02 | rep_origin | 15688 | 16092 | 405 |
| oCOLE-06 | rep_origin | 16770 | 17576 | 807 |

26446 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-F357W | CDS | 2037 | 6062 | 4026 |
| xSV40NLS-06 | misc_signal | 2040 | 2060 | 21 |
| xLinker-06 | misc_feature | 2061 | 2150 | 90 |
| F357W | misc_feature | 3219 | 3221 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27495 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 22018 | 1802 |
| u5SoUbi4-02 | 5'UTR | 596 | 660 | 65 |
| iSoUbi4-02 | intron | 661 | 2018 | 1358 |
| cMb2Cas12a-F547Y | CDS | 2037 | 6062 | 4026 |
| xSV40NLS-06 | misc_signal | 2040 | 2060 | 21 |
| xLinker-06 | misc_feature | 2061 | 2150 | 90 |
| F547Y | misc_feature | 3789 | 3791 | 3 |
| BH | misc_feature | 4863 | 4898 | 36 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 17519 | 1074 |
| oVS1-02 | rep_origin | 15631 | 16035 | 405 |
| oCOLE-06 | rep_origin | 16713 | 15588 | 807 |

27745 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-A742S | CDS | 2037 | 6062 | 4026 |
| xSV40NLS-06 | misc_signal | 2040 | 2060 | 21 |
| xLinker-06 | misc_feature | 2061 | 2150 | 90 |
| A742S | mutation | 4374 | 4376 | 3 |
| BH | misc_feature | 4863 | 4898 | 36 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27747 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-Y819F | CDS | 2037 | 6062 | 4026 |
| xSV40NLS-06 | misc_signal | 2040 | 2060 | 21 |
| xLinker-06 | misc_feature | 2061 | 2150 | 90 |
| Y819F | mutation | 4605 | 4607 | 3 |
| BH | misc_feature | 4863 | 4898 | 36 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27501 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-H939Q | CDS | 2037 | 6062 | 4026 |
| xSV40NLS-06 | misc_signal | 2040 | 2060 | 21 |
| xLinker-06 | misc_feature | 2061 | 2150 | 90 |
| BH | misc_feature | 4863 | 4898 | 36 |
| H939Q | misc_feature | 4965 | 4967 | 3 |
| Junction | misc_feature | 4968 | 4973 | 6 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27218 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+D172R | CDS | 2037 | 6035 | 3999 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| Up-seq-BH2 | misc_feature | 4818 | 5889 | 72 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27025 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc-As+F357W | CDS | 2037 | 6035 | 3999 |
| xSV40NLS-06 | misc_signal | 2040 | 2060 | 21 |
| xLinker-06 | misc_feature | 2061 | 2150 | 90 |
| F357W | misc_feature | 3219 | 3221 | 3 |
| Up-seq-BH2 | misc_feature | 4818 | 4889 | 72 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| OCOLE-06 | rep_origin | 16686 | 17492 | 807 |

27216 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1-As+F357W+D172R | CDS | 2037 | 6035 | 3999 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | misc_feature | 3219 | 3221 | 3 |
| Up-seq-BH2 | misc_feature | 4818 | 4889 | 72 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8285 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27219 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 218 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1-As+-F357W-V921K | CDS | 2037 | 6035 | 3999 |
| xSV40NLS-06 | misc_signal | 2040 | 2060 | 21 |
| xLinker-06 | misc_feature | 2061 | 2150 | 90 |
| F357W | misc_feature | 3219 | 3221 | 3 |
| BH2-As | misc_feature | 4818 | 4889 | 72 |
| V921K | misc_feature | 4884 | 4886 | 3 |
| alpha helix | misc_feature | 5583 | 5627 | 45 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27220 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1-As+-F357W+V921K+D172R | CDS | 2037 | 6035 | 3999 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | misc_feature | 3219 | 3221 | 3 |
| BH2-As | misc_feature | 4818 | 4889 | 72 |
| V921K | misc_feature | 4884 | 4886 | 3 |
| alpha helix | misc_feature | 5583 | 5627 | 45 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17495 | 807 |

27225 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As_Nuc1-As+F357W+V921K+E797A | CDS | 2037 | 6035 | 3999 |
| xSV40NLS-06 | misc_signal | 2040 | 2060 | 21 |
| xLinker-06 | misc_feature | 2061 | 2150 | 90 |
| cMb2Cas12a-a | CDS | 3219 | 3226 | 8 |
| F357W | misc_feature | 3219 | 3221 | 3 |
| xSGGSlinker-06 | misc_feature | 3877 | 3888 | 12 |
| E797A | misc_feature | 4539 | 4541 | 3 |
| BH2-As | misc_feature | 4818 | 4889 | 72 |
| V921K | misc_feature | 4884 | 4886 | 3 |
| alpha helix | misc_feature | 5583 | 5627 | 45 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| rHHMb2CrRNA-01 | misc_RNA | 8110 | 8152 | 43 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-01 | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa-01 | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 1183 | 1143 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27228 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1-As+F357W+ | CDS | 2037 | 6035 | 3999 |
| V921K-+E797A+D172R | | | | |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | misc_feature | 3219 | 3221 | 3 |
| E797A | misc_feature | 4539 | 4541 | 3 |
| BH2-As | misc_feature | 4818 | 4889 | 72 |
| V921K | misc_feature | 4884 | 4886 | 3 |
| alpha helix | misc_feature | 5583 | 5627 | 45 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27223 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1-As+F357W+ | | | | |
| V921K+-E797A+WED-MR1 | CDS | 2037 | 6035 | 3999 |
| xSV40NLS-06 | misc_signal | 2040 | 2060 | 21 |
| xLinker-06 | misc_feature | 2061 | 2150 | 90 |
| F357W | misc_feature | 3219 | 3221 | 3 |
| clbCas12a-05 | misc_feature | 4497 | 4517 | 21 |
| E797A | misc_feature | 4539 | 4541 | 3 |
| BH2-As | misc_feature | 4818 | 4889 | 72 |
| V921K | misc_feature | 4884 | 4886 | 3 |
| alpha helix | misc_feature | 5583 | 5627 | 45 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27224 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1- | CDS | 2037 | 6035 | 3999 |
| As+F357W+V921K+E797A+WED-MR1+D172R | | | | |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | misc_feature | 3219 | 3221 | 3 |
| E797A | misc_feature | 4539 | 4541 | 3 |
| BH2-As | misc_feature | 4818 | 4889 | 72 |
| V921K | misc_feature | 4884 | 4886 | 3 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27310 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+E797A | CDS | 2037 | 6035 | 3999 |
| xSV40NLS-06 | misc_signal | 2040 | 2060 | 21 |
| xLinker-06 | misc_feature | 2061 | 2150 | 90 |
| E797A | mutation | 4539 | 4541 | 3 |
| BH2-As | misc_feature | 4818 | 4889 | 72 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27311 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+E797A+D172R | CDS | 2037 | 6035 | 3999 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| E797A | mutation | 4539 | 4541 | 3 |
| BH2-As | misc_feature | 4818 | 4889 | 72 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27320 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+E797A+F357W | CDS | 2037 | 6035 | 3999 |
| xSV40NLS-06 | misc_signal | 2040 | 2060 | 21 |
| xLinker-06 | misc_feature | 2061 | 2150 | 90 |
| F357W | mutation | 3219 | 3221 | 3 |
| E797A | mutation | 4539 | 4541 | 3 |
| BH2-As | misc_feature | 4818 | 4889 | 72 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27321 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2- | | | | |
| As+E797A+F357W+D172R | CDS | 2037 | 6035 | 3999 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | mutation | 3219 | 3221 | 3 |
| E797A | mutation | 4539 | 4541 | 3 |
| BH2-As | misc_feature | 4818 | 4889 | 72 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 1448 | 15561 | 1074 |
| oVS1-02 | rep_origin | 15604 | 16008 | 405 |
| oCOLE-06 | rep_origin | 16686 | 17492 | 807 |

27322 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-V921K+E797A | CDS | 2037 | 6062 | 4026 |
| xSV40NLS-06 | misc_signal | 2040 | 2060 | 21 |
| xLinker-06 | misc_feature | 2061 | 2150 | 90 |
| E797A | misc_feature | 4539 | 4541 | 3 |
| V921K | misc_feature | 4911 | 4913 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 87 |

27381 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-V921K+E797A+D172R | CDS | 2037 | 6062 | 4026 |
| xSV40NLS-06 | misc_signal | 2040 | 5060 | 21 |
| xLinker-06 | misc_feature | 2061 | 2150 | 90 |
| D172R | mutation | 2664 | 2666 | 3 |
| E797A | mutation | 4539 | 4541 | 3 |
| V921K | misc_feature | 4911 | 4913 | 3 |
| xLinker-06 | misc_feature | 5904 | 5993 | 90 |
| xSV40NLS-04 | misc_signal | 5990 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27382 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-V921K+E797A+F357W | CDS | 2037 | 6062 | 4026 |
| xSV40NLS-06 | misc_signal | 2040 | 2060 | 21 |
| xLinker-06 | misc_feature | 2061 | 2150 | 90 |
| F357W | misc_feature | 3219 | 3221 | 3 |
| E797A | mutation | 4539 | 4541 | 3 |
| V921K | misc_feature | 4911 | 4913 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27383 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-V921K+E797A+F357W+D172R | CDS | 2037 | 6062 | 4026 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | misc_feature | 3219 | 3221 | 3 |
| E797A | misc_feature | 4539 | 4541 | 3 |
| V921K | misc_feature | 4911 | 4913 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8139 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| CPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 1074 |
| oVS1-02 | rep_origin | 15631 | 16035 | 405 |
| oCOLE-06 | rep_origin | 16713 | 17519 | 807 |

27325 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-BH2-As+Nuc1-As+F357W+E797A | CDS | 2037 | 6035 | 3999 |
| xSV40NLS-06 | misc_signal | 2040 | 2060 | 21 |
| xLinker-06 | misc_feature | 2061 | 2150 | 90 |
| F357W | misc_feature | 3219 | 3221 | 3 |
| xSGGSlinker-06 | misc_feature | 3877 | 3888 | 12 |
| E797A | mutation | 4539 | 4541 | 3 |
| BH2-As | misc_feature | 4818 | 4889 | 72 |
| alpha helix | misc_feature | 5583 | 5627 | 45 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| rHHMb2CrRNA-01 | misc_RNA | 8110 | 8152 | 43 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-01 | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa-01 | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| cPMI-01 | CDS | 10658 | 11833 | 1176 |
| tNOS-05-01 | terminator | 11894 | 12146 | 253 |
| bNLB-05 | insertion_seq | 12236 | 12365 | 130 |
| bNLB-01-01 | insertion_seq | 12271 | 12295 | 25 |
| cSpec-03 | CDS | 12645 | 13433 | 789 |
| prVirG-01 | promoter | 13528 | 13658 | 131 |
| cVirG-01 | CDS | 13733 | 14458 | 726 |
| cRepA-03 | CDS | 14488 | 15561 | 1074 |
| oVS1-02 | rep_origin | 15604 | 16008 | 405 |
| oCOLE-06 | rep_origin | 16686 | 17492 | 807 |

27323 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| Mb2Cas12a-BH2-As+Nuc1-As+F357W+E797A+D172R | CDS | 2037 | 6035 | 3999 |
| xSV40NLS-06 | misc_signal | 2040 | 2060 | 21 |
| xLinker-06 | misc_feature | 2061 | 2150 | 90 |
| D172R | misc_feature | 2664 | 2666 | 3 |
| F357W | misc_feature | 3219 | 3221 | 3 |
| E797A | misc_feature | 4539 | 4541 | 3 |
| Up-seq-BH | misc_feature | 4818 | 4889 | 72 |
| Nuclease active site-As | misc_feature | 5628 | 5666 | 39 |
| xLinker-06 | misc_feature | 5877 | 5966 | 90 |
| xSV40NLS-04 | misc_signal | 5967 | 5987 | 21 |
| xSGGSlinker-02 | misc_feature | 5988 | 5999 | 12 |
| xSGGSlinker-02 | misc_feature | 6000 | 6011 | 12 |
| xSV40NLS-07 | misc_signal | 6012 | 6032 | 21 |
| tNOS-05-01 | terminator | 6043 | 6295 | 253 |
| prSoUbi4-02 | promoter | 6302 | 8103 | 1802 |
| u5SoUbi4-02 | 5'UTR | 6681 | 6745 | 65 |
| iSoUbi4-02 | intron | 6746 | 8103 | 1358 |
| pairing\sequence | misc_feature | 8110 | 8115 | 6 |
| rHH-05 | misc_RNA | 8116 | 8152 | 37 |
| rMb2gRNACas12aZmWxy1-01 | misc_RNA | 8153 | 8195 | 43 |
| rMb2CrRNA-01 | misc_RNA | 8153 | 8172 | 20 |
| ZmWxy1\target | misc_feature | 8173 | 8195 | 23 |
| rMb2gRNACas12aZmBX9-A | misc_RNA | 8196 | 8238 | 43 |
| rMb2CrRNA-01 | misc_RNA | 8196 | 8215 | 20 |
| ZmBX9\target | misc_feature | 8216 | 8238 | 23 |
| rMb2gRNACas12aZmGL2-01 | misc_RNA | 8239 | 8282 | 44 |
| rMb2CrRNA-01 | misc_RNA | 8239 | 8258 | 20 |
| ZmGL2\target | misc_feature | 8259 | 8282 | 24 |
| rMb2gRNACas12aZmBINa | misc_RNA | 8283 | 8325 | 43 |
| rMb2CrRNA-01 | misc_RNA | 8283 | 8302 | 20 |
| ZmBINa\target | misc_feature | 8303 | 8325 | 23 |
| rHDV-01 | misc_RNA | 8326 | 8393 | 68 |
| tNOS-05-01 | terminator | 8394 | 8646 | 253 |
| prUbi1-10 | promoter | 8653 | 10645 | 1993 |
| TSS | misc_feature | 9553 | 9553 | 1 |
| iUbi1-02-01 | intron | 9636 | 10645 | 1010 |
| | CDS | 10658 | 11833 | 1176 |
| | terminator | 11894 | 12146 | 253 |
| | insertion_seq | 12236 | 12365 | 130 |
| | insertion_seq | 12271 | 12295 | 25 |
| | CDS | 12645 | 13433 | 789 |
| | promoter | 13528 | 13658 | 131 |
| | CDS | 13733 | 14458 | 726 |
| | CDS | 14488 | 15561 | 1074 |
| | rep_origin | 15604 | 16008 | 405 |
| | rep_origin | 16686 | 17492 | 807 |

27926 Annotations:

| Name | Type | Minimum | Maximum | Length |
|---|---|---|---|---|
| bNRB-04 | insertion_seq | 4 | 143 | 140 |
| bNRB-01-01 | insertion_seq | 101 | 125 | 25 |
| prSoUbi4-02 | promoter | 217 | 2018 | 1802 |
| u5SoUbi4-02 | 5'UTR | 596 | 660 | 65 |
| iSoUbi4-02 | intron | 661 | 2018 | 1358 |
| cMb2Cas12a-1914K+L917V+N918A | CDS | 2037 | 6062 | 4026 |
| xSV40NLS-06 | misc_signal | 2040 | 2060 | 21 |
| xLinker-06 | misc_feature | 2061 | 2150 | 90 |
| 1914K | misc_feature | 4890 | 4892 | 3 |
| L917V | misc_feature | 4899 | 4901 | 3 |
| N918A | misc_feature | 4902 | 4904 | 3 |
| xLinker-06 | misc_feature | 5904 | 5993 | 90 |
| xSV40NLS-04 | misc_signal | 5994 | 6014 | 21 |
| xSGGSlinker-02 | misc_feature | 6015 | 6026 | 12 |
| xSGGSlinker-02 | misc_feature | 6027 | 6038 | 12 |
| xSV40NLS-07 | misc_signal | 6039 | 6059 | 21 |
| tNOS-05-01 | terminator | 6070 | 6322 | 253 |
| prSoUbi4-02 | promoter | 6329 | 8130 | 1802 |
| u5SoUbi4-02 | 5'UTR | 6708 | 6772 | 65 |
| iSoUbi4-02 | intron | 6773 | 8130 | 1358 |
| pairing\sequence | misc_feature | 8137 | 8142 | 6 |
| rHH-05 | misc_RNA | 8143 | 8179 | 37 |
| rMb2gRNACas12aZmWxy1-01 | misc_RNA | 8180 | 8222 | 43 |
| rMb2CrRNA-01 | misc_RNA | 8180 | 8199 | 20 |
| ZmWxy1\target | misc_feature | 8200 | 8222 | 23 |
| rMb2gRNACas12aZmBX9-A | misc_RNA | 8223 | 8265 | 43 |
| rMb2CrRNA-01 | misc_RNA | 8223 | 8242 | 20 |
| ZmBX9\target | misc_feature | 8243 | 8265 | 23 |
| rMb2gRNACas12aZmGL2-01 | misc_RNA | 8266 | 8309 | 44 |
| rMb2CrRNA-01 | misc_RNA | 8266 | 8285 | 20 |
| ZmGL2\target | misc_feature | 8286 | 8309 | 24 |
| rMb2gRNACas12aZmBINa | misc_RNA | 8310 | 8352 | 43 |
| rMb2CrRNA-01 | misc_RNA | 8310 | 8329 | 20 |
| ZmBINa\target | misc_feature | 8330 | 8352 | 23 |
| rHDV-01 | misc_RNA | 8353 | 8420 | 68 |
| tNOS-05-01 | terminator | 8421 | 8673 | 253 |
| prUbi1-10 | promoter | 8680 | 10672 | 1993 |
| TSS | misc_feature | 9580 | 9580 | 1 |
| iUbi1-02-01 | intron | 9663 | 10672 | 1010 |
| cPMI-01 | CDS | 10685 | 11860 | 1176 |
| tNOS-05-01 | terminator | 11921 | 12173 | 253 |
| bNLB-05 | insertion_seq | 12263 | 12392 | 130 |
| bNLB-01-01 | insertion_seq | 12298 | 12322 | 25 |
| cSpec-03 | CDS | 12672 | 13460 | 789 |
| prVirG-01 | promoter | 13555 | 13685 | 131 |
| cVirG-01 | CDS | 13760 | 14485 | 726 |
| cRepA-03 | CDS | 14515 | 15588 | 104 |
| oVS1-02 | rep_origin | 15631 | 16035 | 405 |
| oCOLE-06 | rep_origin | 16713 | 17519 | 807 |

1. A mutant Mb2Cas 12a polypeptide comprising at least one amino acid substitution introduced into a wild-type Mb2Cas 12a polypeptide sequence of SEQ ID NO: 1.

2. The mutant Mb2Cas12a polypeptide of claim 1, wherein the at least one amino acid substitution occurs at a position selected from the group consisting of D172, F357, F547, A742, E797, Y819, E913, 1914, L917, N918, V921, H939, and Y1172.

3. The mutant Mb2Cas12a polypeptide of claim 2, wherein the at least one amino acid substitution is selected from the group consisting of D172R, D172K, F357W, F547Y, A742S, E797A, Y819F, 1914K, L917V, N918A, N918K, V921K, V921Q, H939Q, and Y1172N.

4. The mutant Mb2Cas12a polypeptide of claim 3, wherein the polypeptide comprises a sequence selected from the group comprising SEQ ID NOs: 3, 5, 26, 35, 57, 58, 64, 76, 80, 85, 86, 87, 88, and 127.

5. A mutant Mb2Cas 12a polypeptide comprising at least one domain swap.

6. The mutant Mb2Cas12a polypeptide of claim 5, wherein the domain swap occurs at a domain selected from the group consisting of WED-MR1, WED-MR2, BH, Up-seq, and Nuc.

7. The mutant Mb2Cas12a polypeptide of claim 6, wherein the WED-MR1 domain is replaced with a sequence selected from the group consisting of SEQ ID NOs: 24, 29, and 30.

8. The mutant Mb2Cas12a polypeptide of claim 6, wherein the WED-MR2 domain is replaced with a sequence selected from the group consisting of SEQ ID NOs: 25, 32, and 33.

9. The mutant Mb2Cas12a polypeptide of claim 6, wherein the BH domain is replaced with a sequence selected from the group consisting of SEQ ID NOs: 40, 41, and 42.

10. The mutant Mb2Cas12a polypeptide of claim 6, wherein the Up-seq domain is replaced with a sequence selected from the group consisting of SEQ ID NOs: 59, 60, 61, 62, and 63.

11. The mutant Mb2Cas12a polypeptide of claim 6, wherein the Nuc domain is replaced with a sequence selected from the group consisting of SEQ ID NOs: 65, 66, and 67.

12. The mutant Mb2Cas12a polypeptide of claim 5, wherein the polypeptide comprises a sequence selected from the group comprising SEQ ID NOs: 37, 59, 60, 61, 62, 63, 74, 75, 77, and 78.

13. A mutant Mb2Cas12a polypeptide comprising at least one amino acid substitution and at least one domain swap.

14. The mutant Mb2Cas12a polypeptide of claim 13, wherein the Mb2Cas12a polypeptide sequence is selected from the group consisting of SEQ ID NOs: 36, 89, 90, 91, 92, 93, 94, 95, 96, 97, 98, 99, 100, 101, 102, 103, 104, 105, 106, and 107.

15. A method of editing a plant genome, comprising contacting said plant genome with the mutant Mb2Cas12a polypeptide of claim 1.

16. The method of claim 15, further comprising a guide RNA.

17. The method of claim 16, wherein the guide RNA is encoded by a sequence comprising SEQ ID NOs: 18-21.

18. An edited plant obtained by the method of claim 1.

19. A construct or plasmid comprising a polynucleotide sequence encoding for the mutant Mb2Cas 12a polypeptide of claim 1.

20. A non-human cell comprising the construct or plasmid of claim 19.
